SEQUENCE LISTING (12 sequences)

SEQ ID NO: 1 - protein, 1273 aa, Artificial Sequence
  Spike Glycoprotein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1027 aa, Artificial Sequence
  4MVac Spike-M8 (mutated Spike Glycoprotein)
MFVFLVLLPLVSSQCVNLTTRTTRGVYYPDKVFRSSVLHSTQDGPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLFGSVACTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSANSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 3 - protein, 6 aa, Artificial Sequence
  Amino acid position 611-616 in mutated
LFGSVA

SEQ ID NO: 4 - protein, 13 aa, Artificial Sequence
  Signal sequence
MFVFLVLLPLVSS

SEQ ID NO: 5 - protein, 22 aa, Artificial Sequence
  Signal sequence
MFVFLVLLPLVSSQCVNLTTRT

SEQ ID NO: 6 - protein, 4 aa, Artificial Sequence
  Amino acid position 682-685 in mutated
SGAG

SEQ ID NO: 7 - protein, 6 aa, Artificial Sequence
  Amino acid position 611-616
LYQDVN

SEQ ID NO: 8 - protein, 4 aa, Artificial Sequence
  Amino acid position 682-685
RRAR

SEQ ID NO: 9 - protein, 19 aa, Artificial Sequence
  Deleted sequence 1255-1273
KFDEDDSEPVLKGVKLHYT

SEQ ID NO: 10 - DNA, 20 bp, Artificial Sequence
  CoV_N1 F Primer
gaccccaaaatcagcgaaat

SEQ ID NO: 11 - DNA, 24 bp, Artificial Sequence
  CoV_N1 R primer
tctggttactgccagttgaatctg

SEQ ID NO: 12 - DNA, 24 bp, Artificial Sequence
  CoV_N1 probe
accccgcattacgtttggtggacc

(19) United States
(12) Patent Application Publication
(10) Pub. No.: US 2023/0331782 A1
(43) Pub. Date: Oct. 19, 2023

(54) Compositions and Methods for Reducing Risk of Vaccine-Enhanced Disease

(71) Applicant: The Trustees of the University of Pennsylvania, Philadelphia, PA (US)

(72) Inventor: Hansell Stedman, Norristown, PA (US)

(21) Appl. No.: 18/024,872

(22) PCT Filed: Sep. 8, 2021
(86) PCT No.: PCT/US2021/049438

§ 371 Date: Mar. 6, 2023

Related U.S. Application Data
(60) Provisional application No. 63/075,679, filed on Sep. 8, 2020.

(51) Int. Cl. C07K 14/005; A61K 39/215; A61P 31/14; C12N 15/86

(52) U.S. Cl. CPC C07K 14/005; A61K 39/215; A61P 31/14; C12N 15/86; A61K 2039/53

(57) ABSTRACT

In one aspect, the present disclosure relates to a mutated SARS-CoV-2 S glycoprotein (mutated S glycoprotein) comprising a SARS-CoV-2 S glycoprotein amino acid sequence having one or more mutations compared to a wildtype S glycoprotein, wherein the mutated S glycoprotein minimizes (i) antibody-dependent enhancement (ADE) and/or (ii) vaccine-associated enhanced respiratory disease (VAERD) when administered to or expressed in a subject. In another aspect, the present disclosure relates to a method of using the mutated S glycoprotein of the present disclosure to induce at least partial immunity to a coronavirus in a subject.

### CROSS-REFERENCE TO RELATED APPLICATION

The present application is entitled to priority under 35 U.S.C. § 119(e) to U.S. Provisional Patent Application No. 63/075,679 filed Sep. 8, 2020, which is hereby incorporated by reference in its entirety herein.

### SEQUENCE LISTING

The ASCII text file named “046483_7309WO1SequenceListing” created on Sep. 8, 2021, comprising 22 Kbytes, is hereby incorporated by reference in its entirety.

### BACKGROUND OF THE INVENTION

Development of effective vaccines to extinguish the COVID-19 pandemic will require careful consideration of the risk:benefit calculus as each phase of the research informs the next. A major concern is the potential risk of inciting vaccine-enhanced disease in previously healthy people (Food and Drug Administration et al., “Development and Licensure of Vaccines to Prevent COVID-19, Guidance for Industry,” June 2020, pp. 1-21). Studies of candidate vaccines against several viruses have elucidated mechanisms for two overlapping enhancement syndromes: antibody-dependent enhancement (ADE) and vaccine-associated enhanced respiratory disease (VAERD). A major but not exclusive (Wang, Q. et al., “Immunodominant SARS Coronavirus Epitopes in Humans Elicited both Enhancing and Neutralizing Effects on Infection in Non-human Primates,” ACS Infect. Dis., 2016, 2:361-376) mechanism of ADE is a viral infection of host cells by receptor mediated engagement of virus-bound but non-neutralizing antibodies through the Fc domain (the conserved-sequence C-terminal half of the heavy chain opposite the variable-sequence antigen-binding Fab domain) (Iwasaki, A. et al., “The potential danger of suboptimal antibody responses in COVID-19,” Nat. Rev. Immunol., 2020, 20:339-341). Major mechanisms of VAERD include failure of vaccination, as seen with purified protein or formalin-inactivated virus, to achieve intracellular processing of native antigens so as to include an MHC class I pathway (Browne, S. K. et al., “Summary of the Vaccines and Related Biological Products Advisory Committee meeting held to consider evaluation of vaccine candidates for the prevention of respiratory syncytial virus disease in RSV-naïve infants,” Vaccine, 2020, 38:101-106).

There remains a need in the art for effective vaccines against viruses such as SARS-CoV-2. The present invention satisfies these unmet needs.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIGS. 1A-1B represent CN3D depictions of Spike homotrimer (FIG. 1A) and isolated protomer (FIG. 1B) to illustrate the relative positions of the eight site-directed mutations (numbered 1-8) described elsewhere herein. All mutations/deletions are depicted by changing the shading of the protomer at the indicated sites. Note that the N-terminal domain (NTD) deletions are also illustrated on the protomer to illustrate the removal of the autonomously folding, exposed NTD.

FIG. 2 depicts a rectangle emphasizing the preservation of the interface between the protomers to optimize the chaperone function of the crown during subsequent folding of the portion corresponding to the S2 subfragment.

FIG. 3 depicts an oblique view, from viral protein membrane proximal to distal, illustrating the interface between retained portions of the protomers, and the relative isolation of the deleted NTD from the rest of the protomer, as depicted with different shading.

FIG. 4 depicts an alignment of a sequence of a wildtype SARS-CoV-2 S glycoprotein (“Spike Glycoprot”) (SEQ ID NO: 1) and a sequence of a mutated S glycoprotein (“4MVac Spike-M8”) (SEQ ID NO: 2) containing the eight mutations described elsewhere herein. SEQ ID NO: 1 encodes a wildtype precursor S glycoprotein. The eight mutations are highlighted.

FIG. 5 (left) depicts a Western blot showing expression in HEK293 cells of SARS-CoV-2 Spike protein containing the eight mutations as described elsewhere herein (Spike-M8) and expression of a wildtype SARS-CoV-2 Spike protein as a control. FIG. 5 (right) depicts a Western blot showing expression of the Spike-M8 protein in vivo (mouse limb gastrocnemius muscle) at 7 days following intramuscular injection with the encoding AAV vector.

FIGS. 6A-6D depict the AAV vaccine design and characterization. FIG. 6A depicts a linear schematic of mutations and deletions (D's) present in vaccine candidates M8 and M8B mapped onto spike protein from SARS-CoV-2 strain Wuhan/IVDC-HB-01/2019. SS: Signal sequence. NTD: N-Terminal Domain. RBD: Receptor Binding Domain. TM: Transmembrane Domain. ER: ER Retention Signal. FIG. 6B depicts a schematic depiction of recombinant AAV genome encoding a CMV promoter driven, codon-optimized, truncated spike transgene. FIG. 6C depicts the detection of M8 and M8B by western blot in HEK293 cells transfected with ITR-containing pAAVITRCMV-M8B plasmid. Recombinant spike protein was used as a positive control. FIG. 6D depicts the immunohistochemistry results of C57BL/10 gastrocnemius muscle 7 days post IM vaccination of 6.4E10 vg AAV9-M8B (n=2) as well as an unvaccinated negative control. Wheat germ agglutinin (WGA) demarcates the muscles fiber membranes.

FIGS. 7A-7B depict the in vitro characterization results. In FIG. 7A, c2cl2 cells were CMV-UTRS′-M8B-S′UTR-pA-AAV_ITR and then grown on chamber slides. In FIG. 7B, cells were later fixed and stained for spike protein and nuclei labeled with DAPI. (FIG. 7B).

FIGS. 8A-8G depict AAV vaccine-induced cellular immunogenicity in mice. FIG. 8A and FIG. 8B depict immunohistochemistry results showing F4/80+ macrophage (FIG. 8A) and CD8+ cytotoxic T-cell (FIG. 8B) infiltration surrounding M8B expressing muscle fibers at 3 weeks post IM injection of 6.4E10 vg AAV9-M8B (n=2). Scale bar=100 um. Arrows in the merged images indicate centrally nucleated, actively regenerating muscle fibers (FIG. 8B). In FIG. 8C, C57BL/10 mice received 6.4E10 vg IM injection of either AAV9-M8, AAV9-M8B, AAV6-M8B, or AAV buffer (PBS+0.001% PF68) as a mock vaccinated control. At one month post vaccination, the indicated number of IFNy secreting cells per 1E6 splenocytes were detected by ELISpot in response to ex vivo stimulation with peptide pools spanning both the SI and S2 fragment of WT HB-01 spike, d-g. Mixed gender 2-3-month-old mice received either 1E12 vg AAV6-M8B (n=5 kl8-hACE2 mice) or AAV buffer (n=2 kl8-hACE2, n=3 C57BL/10). At 65 days post vaccination, the indicated number of IFNy (FIG. 8D), IL-2 (FIG. 8E), IL-4 (FIG. 8F), and IL-10 (FIG. 8G) secreting cells per 1E6 splenocytes were detected by ELISpot in response to ex vivo stimulation with individual peptide pools spanning the S1 and S2 fragment of WT HB-01 spike. Alpha variant spike peptide pools were used in FIG. 8D where indicated, (FIG. 8C to FIG. 8G). Symbols represent individual animals. Lines show mean+/−1 S.D. Results were compared either by Brown-Forsythe and Welch's analysis of variance (ANOVA) with Dunnett's T3 multiple comparisons test (FIG. 8C and FIG. 8D) or Welch's t-test (FIG. 8E to FIG. 8G).

FIGS. 9A-9B depict that AAV vaccine immunogenicity is directed against foreign M8/M8B spike protein, not AAV capsid. F4/80+ macrophages (FIG. 9A) and CD8+ cytotoxic T-cells (FIG. 9B) fail to infiltrate and surround Utrophin expressing muscle fibers at 3 weeks post 50 ul IM injection of 6.4E10 vg. AAV9-Utrophin.

FIGS. 10A-10I depict that AAV-M8/M8B protect transgenic k18-hACE2 mice from lethal SARS-CoV-2 challenge and associated severe disease. FIG. 10A-FIG. 10I depict k18-hACE2 mice receiving an IM injection of either 1E12 vg AAV6-M8B (n=5), 1E12 vg AAV9-M8B (n=5), 1E12 AAV9-M8 (n=5), or AAV formulation buffer (n=5) as a mock vaccination control. Genders within groups were randomized with either 2 males/3 females, or the inverse. FIG. 10A depicts the weight trace post challenge. Groups were compared by two-way ANOVA with Dunnett's multiple comparisons test, p-values shown in figure are at 6 dpi compared to mock. At 5 dpi, p<0.01 for AAV9-M8b and p<0.001 for AAV6-M8B and AAV9-M8. FIG. 10B depicts the results of a treadmill performance test at 5 dpi, as measured by distance traveled in 8 min. FIG. 10C and FIG. 10D depict the results of the respiratory assessment at both 5 and 6 dpi, as measured by tidal volume (FIG. 10C) and respiratory rate (FIG. 10D). Days 5 and 6 were independently compared by Brown-Forsythe and Welch's analysis of variance (ANOVA) with Dunnett's T3 multiple comparisons test. FIG. 10E depict the representative lung histopathology. Scale bar=100 um. FIG. 10F depict viral RNA quantified from whole lung homogenates. FIG. 10G depicts Anti-Spike RBD IgG titers determined by ELISA following viral challenge compared to a group of 1E12 vg AAV6-M8B vaccinated, but unchallenged mice (n=6) at the equivalent timepoint post-vaccination. FIG. 10H depicts SARS-CoV-2 neutralizing antibody titers determined by focus reduction neutralization test (FRNT₅₀). This was similarly compared to the unchallenged AAV6-M8B vaccinated group as in FIG. 10G. FIG. 10I depicts the Kaplan Meier curve showing aggregate survival for all unvaccinated and 1E12 vg AAV vaccinated k18-hACE2 mice post infection with 2.5E4 PFU SARS-CoV-2 (AAV6-M8B n=15, AAV9-M8B n=5, AAV9-M8 n=5). Data generated from 3 independent experiments. Survival was compared by the log-ranked Mantel-Cox test, (FIG. 10B to FIG. 10D) Each shape indicates an individual mouse, and the lines show the mean+/−1 S.D. (FIG. 10G) Symbols represent the geometric mean for all replicates of an individual animal, (FIG. 10F-FIG. 10H) the dotted line indicates the limit of detection, and the lines show the mean+/−1 S.D. Groups were compared by Kruskal-Wallis analysis of variance (ANOVA) with Dunn's multiple comparisons test.

FIGS. 11A-11E depict the protection and efficacy in kl8-hACE2 mice. FIG. 11A depicts an experimental schematic for data depicted in FIG. 10. FIG. 11B depicts the Kaplan Meier survival curve. FIG. 11C depicts the individual animal weight traces for each animal following 2.5E4 PFU SARS-CoV-2 IN inoculation. FIG. 11D depicts individual results of the 8-minute treadmill performance test depicted in FIG. 10B. Because there is a maximum performance on the time restricted protocol, if multiple animals achieve a maximal distance, they appear on top of each other. All animals are represented in the graphic. FIG. 11E depicts the lung IHC post SARS-CoV-2 challenge of mice depicted in the schematic in FIG. 11A for spike protein, WGA (extracellular matrix stain to assess gross lung morphology), and DAPI. In FIG. 11C and FIG. 11D the circles represent females and squares represent males.

FIGS. 12A-12E depicts the AAV vaccine immunogenicity in macaques. FIG. 12A depicts the immunohistochemistry results showing CD8+ cytotoxic T cell infiltration surrounding M8B expressing muscle fibers 29 days after a single IM vaccination with either 1E12 vg of AAV6-M8B or AAV9-M8B. FIG. 12B depicts the immunohistochemistry results showing CDllb+ macrophages in the areas nearby CD8+ cytotoxic T cells surrounding muscle fibers following IM vaccination. FIG. 12C depicts the number of IFNy secreting cells per 1E6 PBMCs as detected by ELISpot. FIG. 12D depicts the anti-Spike ECD IgG titers determined by ELISA. FIG. 12E depicts SARS-CoV-2 neutralizing antibody titers determined by focus reduction neutralization test (FRNT₅₀).

FIG. 13 depicts that boosting fails to elicit a neutralizing humoral following booster vaccination. Two to five month-old C57BL/10 mice received 6.4E10 vg IM injection of either AAV9-M8 (n=5) or AAV9-M8B (n=3) and then received an identical booster vaccination 5 weeks later. Three weeks after the booster dose animals were evaluated for the presence of SARS-CoV-2 neutralizing antibodies. Data is reported as log₁₀(inverse SARS-CoV-2 50% pseudovirus-neutralization titer as measured by focus reduction neutralization test (FRNT50)).

FIGS. 14A-14G depict the long term efficacy studies of AAV6-M8B in kl8-hACE2 mice. FIG. 14A depicts a schematic of the experimental plan. A vial of vector was thawed and immediately used to vaccinate “cohort 1” of kl8-hACE2 mice (n=5 AAV6-M8B, n=5 mock). The tube was then kept in at 4° C. for >4 months before it was used to vaccinate a second cohort, “cohort 2,” of kl8-hACE2 mice (n=5 AAV6-M8B, n=6 mock). Both cohorts were challenged days apart from each other with IN inoculation of 2.5E4 PEL) SARS-CoV-2, at 4.5 mpv for cohort 1 (FIG. 14B to FIG. 14D) and at 18 dpv for cohort 2 (FIG. 14E to FIG. 14G). All animals were tracked until a terminal concluding date. FIGS. 14B and 14E depict Kaplan Meier survival curves post SARS-CoV-2 infection. Survival was compared by the log-ranked Mantel-Cox test. FIGS. 14C and 14F depict changes in body weight post SARS-CoV-2 infection. Weight was compared by the nonparametric Mann-Whitney U-test. The shading of the average mock vaccinated body weight in FIG. 14F changes when the entire average weight is derived from a single surviving animal. FIGS. 14D and 14G depict individual animal traces for animals depicted in FIGS. 14C and 14F.

### SUMMARY OF THE INVENTION

As described herein the present invention relates to effective vaccine platforms, that can provide long-term efficacy through the induction of cellular immunity.

In one aspect the invention provides a mutated SARS-CoV-2 S glycoprotein (mutated S glycoprotein) comprising a SARS-CoV-2 S glycoprotein amino acid sequence having one or more mutations compared to a wildtype S glycoprotein, wherein the mutated S glycoprotein minimizes (i) antibody-dependent enhancement (ADE) and/or (ii) vaccine-associated enhanced respiratory disease (VAERD) when administered to or expressed in a subject.

In certain embodiments, the mutated S glycoprotein comprises a sequence having at least 70% sequence identity to SEQ ID NO: 1 and one or more of: (i) a deletion of one or more amino acids from a region spanning amino acid position 14 to amino acid position 32 of a wildtype precursor S glycoprotein of SEQ ID NO: 1; (ii) a deletion of one or more amino acids from within an N-terminal domain of a wildtype precursor S glycoprotein of SEQ ID NO: 1, wherein the deletion removes epitopes in the wildtype mature S glycoprotein that contribute to ADE or VAERD; (iii) an amino acid substitution at amino acid position 271 in a wildtype precursor S glycoprotein of SEQ ID NO: 1; (iv) one or more amino acid substitutions in a region spanning amino acid position 611 to amino acid position 616 of wildtype precursor S glycoprotein of SEQ ID NO: 1; (v) one or more amino acid substitutions in a region spanning amino acid position 682 to amino acid position 685 of wildtype precursor S glycoprotein of SEQ ID NO: 1; (vi) an amino acid substitution at amino acid position 814 and/or amino acid position 815 of wildtype precursor S glycoprotein of SEQ ID NO: 1; (vii) an amino acid substitution at amino acid position 986 and/or amino acid position 987 of wildtype precursor S glycoprotein of SEQ ID NO: 1; and (viii) a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell.

In certain embodiments, the mutated S glycoprotein comprises a signal sequence.

In certain embodiments, the mutated S glycoprotein comprises a deletion of each of the amino acids in the region spanning amino acid position 14 to amino acid position 32 of a wildtype precursor S glycoprotein of SEQ ID NO: 1.

In certain embodiments, the mutated S glycoprotein comprises a deletion of one or more amino acids from a region spanning amino acid position 24 to amino acid position 270 of a wildtype precursor S glycoprotein of SEQ ID NO: 1.

In certain embodiments, the mutated S glycoprotein comprises a deletion of each of the amino acids from the region spanning amino acid position 24 to amino acid position 270 of a wildtype precursor S glycoprotein of SEQ ID NO: 1.

In certain embodiments, the mutated S glycoprotein comprises an amino acid substitution at amino acid position 271 of wildtype precursor S glycoprotein of SEQ ID NO: 1 selected from the group consisting of: Q271G, Q271A, Q271I, Q271L, Q271M, Q271F, Q271P, and Q271V.

In certain embodiments, the mutated S glycoprotein comprises a contiguous amino acid sequence of LFGSVA (SEQ ID NO: 3) at a region corresponding to amino acid position 611 to amino acid position 616 of wildtype precursor S glycoprotein SEQ ID NO: 1.

In certain embodiments, the mutated S glycoprotein comprises a contiguous sequence of SGAG (SEQ ID NO: 6) at a region corresponding to amino acid position 682 to amino acid position 685 of wildtype precursor S glycoprotein of SEQ ID NO: 1.

In certain embodiments, the mutated S glycoprotein comprises a contiguous sequence of AN at a region corresponding to amino acid position 814 to amino acid position 815 of wildtype precursor S glycoprotein of SEQ ID NO: 1.

In certain embodiments, the mutated S glycoprotein comprises a contiguous sequence of PP at a region corresponding to amino acid position 986 to amino acid position 987 of wildtype precursor S glycoprotein of SEQ ID NO: 1.

In certain embodiments, the mutated S glycoprotein comprises a deletion of each of the amino acids in a region extending from amino acid position 1255 to amino acid position 1273 of wildtype precursor S glycoprotein of SEQ ID NO: 1.

In certain embodiments, the mutated S glycoprotein comprises a sequence having at least 70% sequence identity to SEQ ID NO: 1 and a, wherein the mutated S glycoprotein comprises a sequence having at least 80% sequence identity to SEQ ID NO: 2.

In one aspect, the mutated SARS-CoV-2 S glycoprotein (mutated S glycoprotein) comprises a sequence that is at least 80% identical to SEQ ID NO: 2, wherein the mutated SARS-CoV-2 S glycoprotein minimizes (i) antibody-dependent enhancement (ADE) and/or (ii) vaccine-associated enhanced respiratory disease (VAERD) when administered to or expressed in a subject.

In certain embodiments, the mutated S glycoprotein comprises a sequence at least 90% identical to SEQ ID NO: 2.

In certain embodiments, the mutated S glycoprotein comprises a sequence at least 95% identical to SEQ ID NO: 2.

In certain embodiments, the mutated S glycoprotein comprises SEQ ID NO: 2.

In one aspect, the invention provides a pharmaceutical composition comprising a mutated SARS-CoV-2 S glycoprotein (mutated S glycoprotein) comprising a SARS-CoV-2 S glycoprotein amino acid sequence having one or more mutations compared to a wildtype S glycoprotein, wherein the mutated S glycoprotein minimizes (i) antibody-dependent enhancement (ADE) and/or (ii) vaccine-associated enhanced respiratory disease (VAERD) when administered to or expressed in a subject.

In another aspect, the invention provides a pharmaceutical composition comprising a mutated S glycoprotein comprising a sequence having at least 70% sequence identity to SEQ ID NO: 1 and one or more of: (i) a deletion of one or more amino acids from a region spanning amino acid position 14 to amino acid position 32 of a wildtype precursor S glycoprotein of SEQ ID NO: 1; (ii) a deletion of one or more amino acids from within an N-terminal domain of a wildtype precursor S glycoprotein of SEQ ID NO: 1, wherein the deletion removes epitopes in the wildtype mature S glycoprotein that contribute to ADE or VAERD; (iii) an amino acid substitution at amino acid position 271 in a wildtype precursor S glycoprotein of SEQ ID NO: 1; (iv) one or more amino acid substitutions in a region spanning amino acid position 611 to amino acid position 616 of wildtype precursor S glycoprotein of SEQ ID NO: 1; (v) one or more amino acid substitutions in a region spanning amino acid position 682 to amino acid position 685 of wildtype precursor S glycoprotein of SEQ ID NO: 1; (vi) an amino acid substitution at amino acid position 814 and/or amino acid position 815 of wildtype precursor S glycoprotein of SEQ ID NO: 1; (vii) an amino acid substitution at amino acid position 986 and/or amino acid position 987 of wildtype precursor S glycoprotein of SEQ ID NO: 1; and (viii) a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell.

In certain embodiments, the pharmaceutical composition further comprises an adjuvant.

In one aspect, the invention provides a vaccine composition comprising a mutated SARS-CoV-2 S glycoprotein (mutated S glycoprotein) comprising a SARS-CoV-2 S glycoprotein amino acid sequence having one or more mutations compared to a wildtype S glycoprotein, wherein the mutated S glycoprotein minimizes (i) antibody-dependent enhancement (ADE) and/or (ii) vaccine-associated enhanced respiratory disease (VAERD) when administered to or expressed in a subject.

In another aspect, the invention provides a vaccine composition comprising a mutated S glycoprotein comprising a sequence having at least 70% sequence identity to SEQ ID NO: 1 and one or more of: (i) a deletion of one or more amino acids from a region spanning amino acid position 14 to amino acid position 32 of a wildtype precursor S glycoprotein of SEQ ID NO: 1; (ii) a deletion of one or more amino acids from within an N-terminal domain of a wildtype precursor S glycoprotein of SEQ ID NO: 1, wherein the deletion removes epitopes in the wildtype mature S glycoprotein that contribute to ADE or VAERD; (iii) an amino acid substitution at amino acid position 271 in a wildtype precursor S glycoprotein of SEQ ID NO: 1; (iv) one or more amino acid substitutions in a region spanning amino acid position 611 to amino acid position 616 of wildtype precursor S glycoprotein of SEQ ID NO: 1; (v) one or more amino acid substitutions in a region spanning amino acid position 682 to amino acid position 685 of wildtype precursor S glycoprotein of SEQ ID NO: 1; (vi) an amino acid substitution at amino acid position 814 and/or amino acid position 815 of wildtype precursor S glycoprotein of SEQ ID NO: 1; (vii) an amino acid substitution at amino acid position 986 and/or amino acid position 987 of wildtype precursor S glycoprotein of SEQ ID NO: 1; and (viii) a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell.

In certain embodiments, the vaccine further comprises an adjuvant.

In one aspect, the invention provides a vector comprising a nucleic acid sequence encoding a mutated SARS-CoV-2 S glycoprotein (mutated S glycoprotein) comprising a SARS-CoV-2 S glycoprotein amino acid sequence having one or more mutations compared to a wildtype S glycoprotein, wherein the mutated S glycoprotein minimizes (i) antibody-dependent enhancement (ADE) and/or (ii) vaccine-associated enhanced respiratory disease (VAERD) when expressed in a subject.

In certain embodiments, the vector encodes a mutated S glycoprotein comprising a sequence having at least 70% sequence identity to SEQ ID NO: 1 and one or more of: (i) a deletion of one or more amino acids from a region spanning amino acid position 14 to amino acid position 32 of a wildtype precursor S glycoprotein of SEQ ID NO: 1; (ii) a deletion of one or more amino acids from within an N-terminal domain of a wildtype mature S glycoprotein of SEQ ID NO: 2, wherein the deletion removes epitopes in the wildtype mature S glycoprotein that contribute to ADE or VAERD; (iii) an amino acid substitution at amino acid position 271 in a wildtype precursor S glycoprotein of SEQ ID NO: 1; (iv) one or more amino acid substitutions in a region spanning amino acid position 611 to amino acid position 616 of wildtype precursor S glycoprotein of SEQ ID NO: 1; (v) one or more amino acid substitutions in a region spanning amino acid position 682 to amino acid position 685 of wildtype precursor S glycoprotein of SEQ ID NO: 1; (vi) an amino acid substitution at amino acid position 814 and/or amino acid position 815 of wildtype precursor S glycoprotein of SEQ ID NO: 1; (vii) an amino acid substitution at amino acid position 986 and/or amino acid position 987 of wildtype precursor S glycoprotein of SEQ ID NO: 1; and (viii) a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell.

In certain embodiments, the vector encodes a mutated S glycoprotein comprising a signal sequence.

In certain embodiments, the vector encodes a mutated S glycoprotein comprising a deletion of each of the amino acids in the region spanning amino acid position 14 to amino acid position 32 of a wildtype mature S glycoprotein of SEQ ID NO: 1.

In certain embodiments the vector encodes a mutated S glycoprotein comprising a deletion of one or more amino acids from a region spanning amino acid position 24 to amino acid position 270 of a wildtype precursor S glycoprotein of SEQ ID NO: 1.

In certain embodiments, the vector encodes a mutated S glycoprotein comprising deletion of each of the amino acids from the region spanning amino acid position 24 to amino acid position 270 of a wildtype precursor S glycoprotein of SEQ ID NO: 1.

In certain embodiments, the vector encodes a mutated S glycoprotein comprising an amino acid substitution, wherein the amino acid substitution at amino acid position 271 of wildtype precursor S glycoprotein of SEQ ID NO: 1 is selected from the group consisting of: Q271G, Q271A, Q271I, Q271L, Q271M, Q271F, Q271P, and Q271V.

In certain embodiments, the vector encodes a mutated S glycoprotein comprising a contiguous amino acid sequence of LFGSVA (SEQ ID NO: 3) at a region corresponding to amino acid position 611 to amino acid position 616 of wildtype precursor S glycoprotein of SEQ ID NO: 1.

In certain embodiments, the vector encodes a mutated S glycoprotein comprising a contiguous sequence of SGAG (SEQ ID NO: 6) at a region corresponding to amino acid position 682 to amino acid position 685 of wildtype precursor S glycoprotein of SEQ ID NO: 1.

In certain embodiments, the vector encodes a mutated S glycoprotein comprising a contiguous sequence of AN at a region corresponding to amino acid position 814 to amino acid position 815 of wildtype precursor S glycoprotein of SEQ ID NO: 1.

In certain embodiments, the vector encodes a mutated S glycoprotein comprising a contiguous sequence of PP at a region corresponding to amino acid position 986 to amino acid position 987 of wildtype precursor S glycoprotein of SEQ ID NO: 1.

In certain embodiments, the vector encodes a mutated S glycoprotein comprising a deletion of each of the amino acids in a region extending from amino acid position 1255 to amino acid position 1273 of wildtype precursor S glycoprotein of SEQ ID NO: 1.

In certain embodiments, the vector encodes a mutated S glycoprotein, wherein the mutated S glycoprotein comprises a sequence having at least 80% sequence identity to SEQ ID NO: 2.

In certain embodiments, the vector encodes a mutated S glycoprotein, comprising a promoter operatively linked to the nucleic acid sequence encoding the mutated S glycoprotein.

In certain embodiments, the vector encodes a mutated S glycoprotein comprising a promoter that is a muscle-specific promoter.

In certain embodiments, the vector comprises a muscle-specific promoter selected from the group consisting of: skeletal β-actin, myosin light chain 2A, dystrophin, muscle creatine kinase, SPc-512, and synthetic muscle promoters.

In certain embodiments, the promoter is selected from the group consisting of: CMV, RSV, SV40, β-actin, PGK, and EF1 promoters.

In certain embodiments, the vector is a viral vector.

In certain embodiments, the vector is a lentivirus vector or herpes virus vector.

In certain embodiments, the vector is an AAV vector.

In certain embodiments, the AAV vector comprises an AAV serotype 6 (AAV6) capsid protein.

In certain embodiments, the AAV vector comprises an AAV serotype 9 (AAV9) capsid protein.

In certain embodiments, the AAV vector comprises an Anc80, Anc80Lib, Anc 81, Anc82, Anc83, Anc84, Anc110, Anc113, Anc126, Anc127, or another phylogenetically related AAV capsid protein.

In certain embodiments, the vector is a plasmid.

In certain embodiments, the invention provides a pharmaceutical composition comprising a vector.

In certain embodiments, the pharmaceutical composition further comprises an adjuvant.

In certain embodiments, the adjuvant is a CpG adjuvant.

In certain embodiments, the invention provides a vaccine composition comprising a vector.

In certain embodiments, the vaccine further comprises an adjuvant.

In one aspect, the inventions provides a method of inducing at least partial immunity to a coronavirus in a subject, the method comprising administering to the subject a therapeutically effective amount of a mutated S glycoprotein of any one of claims 1-17, a pharmaceutical composition of any one of claims 18, 19, and 46-48, a vector of any one of claims 22-45, or a vaccine composition of any one of claims 20, 21, and 49-51.

In certain embodiments, the administering of a therapeutically effective amount of a mutated S glycoprotein minimizes antibody-dependent enhancement (ADE).

In certain embodiments, the administering of a therapeutically effective amount of a mutated S glycoprotein minimizes vaccine-associated enhanced respiratory disease (VAERD).

In certain embodiments, the administering of a therapeutically effective amount of a mutated S glycoprotein results in at least partial immunity to the coronavirus due to humoral immunity to the coronavirus.

In certain embodiments, the administering of a therapeutically effective amount of a mutated S glycoprotein results in T-cell mediated immunity to the coronavirus.

In certain embodiments, the administering of a therapeutically effective amount of a mutated S glycoprotein results in an increase in titer of antibodies that specifically bind to the mutated S glycoprotein in the subject.

In certain embodiments, the administering of a therapeutically effective amount of a mutated S glycoprotein results in a decrease in the rate of infection of the coronavirus in the subject.

In certain embodiments, the method further comprises administering an adjuvant to the subject.

In certain embodiments, the subject has been identified as not having previously had a coronavirus infection.

In certain embodiments, prior to the administering step, the subject has been identified as not having a significant titer of antibodies that bind specifically to the S glycoprotein of the fragment thereof.

In certain embodiments, the coronavirus is SARS-CoV-2.

In certain embodiments, the subject has been previously identified as having one or more medical conditions selected from the group consisting of: chronic lung disease, moderate asthma, severe asthma, heart conditions, diabetes, obesity, liver disease, chronic kidney disease, and a weakened or suppressed immune system.

In certain embodiments, the subject having a weakened or suppressed immune system is a subject receiving a cancer treatment, a smoker, a subject who is a transplant recipient, a subject having HIV or AIDS, or a subject receiving a corticosteroid or any other immunosuppressant drug.

In certain embodiments, the subject having a weakened or suppressed immune system is an elderly subject.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

The term “antibody” or “Ab” as used herein, refers to a protein, or polypeptide sequence derived from an immunoglobulin molecule, which specifically binds to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426). An antibody may be derived from natural sources or from recombinant sources. Antibodies are typically tetramers of immunoglobulin molecules.

The term “ameliorating” or “treating” means that the clinical signs and/or the symptoms associated with a disease are lessened as a result of the actions performed. The signs or symptoms to be monitored will be well known to the skilled clinician.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term “biological” or “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

As used herein, the terms “control,” or “reference” are used interchangeably and refer to a value that is used as a standard of comparison.

The term “immunogenicity” as used herein, refers to the innate ability of an antigen or organism to elicit an immune response in an animal when the antigen or organism is administered to the animal. Thus, “enhancing the immunogenicity” refers to increasing the ability of an antigen or organism to elicit an immune response in an animal when the antigen or organism is administered to an animal. The increased ability of an antigen or organism to elicit an immune response can be measured by, among other things, a greater number of antibodies that bind to an antigen or organism, a greater diversity of antibodies to an antigen or organism, a greater number of T-cells specific for an antigen or organism, a greater cytotoxic or helper T-cell response to an antigen or organism, a greater expression of cytokines in response to an antigen, and the like.

As used herein, the terms “eliciting an immune response” or “immunizing” refer to the process of generating a B cell and/or a T cell response against a heterologous protein.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Heterologous antigens” used herein to refer to an antigen that is not endogenous to the organism comprising or expressing an antigen. As an example, a virus vaccine vector comprising or expressing a viral or tumor antigen comprises a heterologous antigen. The term “Heterologous protein” as used herein refers to a protein that elicits a beneficial immune response in a subject (i.e. mammal), irrespective of its source.

The term “specifically binds”, “selectively binds” or “binding specificity” refers to the ability of the humanized antibodies or binding compounds of the invention to bind to a target epitope with a greater affinity than that which results when bound to a non-target epitope. In certain embodiments, specific binding refers to binding to a target with an affinity that is at least 10, 50, 100, 250, 500, or 1000 times greater than the affinity for a non-target epitope.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime.

“Humoral immunity” or “humoral immune response” both refer to B-cell mediated immunity and are mediated by highly specific antibodies, produced and secreted by B-lymphocytes (B-cells).

“Prevention” refers to the use of a pharmaceutical compositions for the vaccination against a disorder.

“Adjuvant” refers to a substance that is capable of potentiating the immunogenicity of an antigen. Adjuvants can be one substance or a mixture of substances and function by acting directly on the immune system or by providing a slow release of an antigen. Examples of adjuvants are aluminium salts, polyanions, bacterial glycopeptides and slow release agents as Freund's incomplete.

“Delivery vehicle” refers to a composition that helps to target the antigen to specific cells and to facilitate the effective recognition of an antigen by the immune system. The best-known delivery vehicles are liposomes, virosomes, microparticles including microspheres and nanospheres, polymers, bacterial ghosts, bacterial polysaccharides, attenuated bacteria, virus like particles, attenuated viruses and ISCOMS.

The term “expression” as used herein is defined as the transcription and/or translation of a particular nucleotide sequence driven by its promoter.

As used herein, the term “expression cassette” means a nucleic acid sequence capable of directing the transcription and/or translation of a heterologous coding sequence. In some embodiments, the expression cassette comprises a promoter sequence operably linked to a sequence encoding a heterologous protein. In some embodiments, the expression cassette further comprises at least one regulatory sequence operably linked to the sequence encoding the heterologous protein.

“Incorporated into” or “encapsulated in” refers to an antigenic peptide that is within a delivery vehicle, such as microparticles, bacterial ghosts, attenuated bacteria, virus like particles, attenuated viruses, ISCOMs, liposomes and preferably virosomes.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “fusion protein” as used herein refers to a protein wherein the protein comprises two or more proteins linked together by peptide bonds or other chemical bonds. The proteins can be linked together directly by a peptide or other chemical bond, or with one or more amino acids between the two or more proteins, referred to herein as a spacer.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “RNA” as used herein is defined as ribonucleic acid.

“Transform”, “transforming”, and “transformation” is used herein to refer to a process of introducing an isolated nucleic acid into the interior of an organism.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

The term “equivalent,” when used in reference to nucleotide sequences, is understood to refer to nucleotide sequences encoding functionally equivalent polypeptides. Equivalent nucleotide sequences will include sequences that differ by one or more nucleotide substitutions, additions- or deletions, such as allelic variants; and will, therefore, include sequences that differ from the nucleotide sequence of the nucleic acids described herein due to the degeneracy of the genetic code.

The term “isolated” as used herein with respect to nucleic acids, such as DNA or RNA, refers to molecules separated from other DNAs or RNAs, respectively that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments, which are not naturally occurring as fragments and would not be found in the natural state. The term “isolated” is also used herein to refer to polypeptides, which are isolated from other cellular proteins and is meant to encompass both purified and recombinant polypeptides. An “isolated cell” or “isolated population of cells” is a cell or population of cells that is not present in its natural environment.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

A “mutation” as used herein is a change in a DNA or amino acid sequence resulting in an alteration from its natural state. A mutation in a DNA sequence can comprise a deletion and/or insertion and/or duplication and/or substitution of at least one deoxyribonucleic acid base such as a purine (adenine and/or thymine) and/or a pyrimidine (guanine and/or cytosine) as compared to a reference sequence, e.g., a wildtype DNA sequence. A mutation in a protein or polypeptide sequence can comprises a deletion, insertion, or substitution of at least one amino acid residue, as compared to a reference sequence, e.g., a wildtype protein sequence.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids. As used herein, nucleic acids include but are not limited to, all nucleic acid sequences which are obtained by any means available in the art, including, without limitation, recombinant means, i.e., the cloning of nucleic acid sequences from a recombinant library or a viral genome, using ordinary cloning technology and PCR™, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. There are numerous expression control sequences, including promoters which are native, constitutive, inducible and/or tissue-specific, are known in the art that may be used in the compositions of the invention. “Operably linked” should be construed to include RNA expression and control sequences in addition to DNA expression and control sequences.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence, which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “effective amount” or “therapeutically effective amount” means the amount of the virus like particle generated from vector of the invention which is required to prevent the particular disease condition, or which reduces the severity of and/or ameliorates the disease condition or at least one symptom thereof or condition associated therewith.

A “subject” or “patient,” as used herein, may be a human or non-human mammal. Non-human mammals include, for example, livestock and pets, such as ovine, bovine, porcine, canine, feline and murine mammals. In some embodiments, the subject is human. In some embodiments, the subject is a domestic pet or livestock.

“Titers” are numerical measures of the concentration of a virus or viral vector compared to a reference sample, where the concentration is determined either by the activity of the virus, or by measuring the number of viruses in a unit volume of buffer. The titer of viral stocks are determined, e.g., by measuring the infectivity of a solution or solutions (typically serial dilutions) of the viruses, e.g., on HeLa cells using the soft agar method (see, Graham & Van Der eb (1973) Virology 52:456-467) or by monitoring resistance conferred to cells, e.g., G418 resistance encoded by the virus or vector, or by quantitating the viruses by UV spectrophotometry (see, Chardonnet & Dales (1970) Virology 40:462-477).

“Vaccination” refers to the process of inoculating a subject with an antigen to elicit an immune response in the subject, that helps to prevent or treat the disease or disorder the antigen is connected with. The term “immunization” is used interchangeably herein with vaccination.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In the present disclosure, the term “vector” includes an autonomously replicating virus.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The technology described herein is designed to achieve durable, protective immune responses to SARS-CoV-2, while minimizing the risk of ADE and VAERD by all of the mechanisms discussed elsewhere herein. The emphasis is on presenting the immune system with a highly expressed, stable, natively configured “pre-fusion” Spike antigen derived by site-directed mutagenesis of a synthetic transgene encoding the SARS-CoV-2 Spike protein. In the derivative Spike transgene, up to eight mutations in cis, including deletions and substitutions, should synergistically achieve the desired outcomes. Delivery of the transgene into host cells by focal administration should insure intracellular processing.

The essential, initial step in host cell infection by most viruses involves the transfer of nucleic acids from the virus interior to the cellular interior. For enveloped viruses, the signature event is membrane fusion, as driven in the case of SARS-CoV-2 and related coronaviruses by the Spike protein after receptor engagement. A majority of published studies have identified the Spike protein as the primary target of neutralizing antibodies, with attention focused on the outermost receptor binding domain. The best characterized mode of neutralization is interference with receptor binding (ACE-2 in the case of SARS-CoV-2), usually as a result of overlap between the epitopes recognized by the antibody and the amino acid side chains in the high affinity receptor binding pocket. Neutralizing antibodies to the highly conserved, virus membrane-proximal “stem” portion of the related MERS coronavirus have been localized, suggesting that the mode of interference is the prevention of Spike refolding. No mechanism has been established for neutralization by any of the rare antibodies targeting the N-terminal domain, suggesting that most such antibodies are non-neutralizing and hence pose a risk for ADE/VAERD. Arguably the single best characterized ADE epitope in any coronavirus is a six amino acid region in SARS CoV (Wang, Q. et al., “Immunodominant SARS Coronavirus Epitopes in Humans Elicited both Enhancing and Neutralizing Effects on Infection in Non-human Primates,” ACS Infect. Dis., 2016, 2:361-376), homologous to positions 611-616 (LYQDVN; SEQ ID NO: 7) in SARS-CoV-2. Antibodies to this immunodominant peptide sequence were repeatedly associated with ADE/VAERD in rhesus macaques.

CoV Spike is a class I fusion protein (Bosch, B. J. et al., “The Coronavirus Spike Protein Is a Class I Virus Fusion Protein: Structural and Functional Characterization of the Fusion Core Complex,” J. Virology, 2003, 77:8801-8811) which utilizes a variety of host cell membrane associated targets to achieve high affinity binding, followed by internal destabilization and triggering of a “spring-loaded” mechanism to enable cell membrane penetration with a fusion polypeptide. High resolution structures of a wide range of class I fusion proteins, including most recently SARS-CoV-2, have revealed both conserved and species-specific attributes. In common with distant homologs from HIV, Ebola, and influenza, the CoV Spike proteins are encoded and assembled as membrane-spanning homotrimers. To achieve fusion competence, all of the class I fusion proteins must undergo proteolytic processing. SARS-CoV-2 Spike has a unique furin site between the two major subfragments, S1 and S2. In view of the ubiquity of furin, the existence of this site has been suggested to contribute to the contagiousness of COVID-19 as proteolysis at this site primes the protein for fusion. Proteolysis at a second site within S2, designated S2′, is also essential to create a metastable protein capable of reconfiguration upon receptor engagement. Tectonic conformational change to achieve fusion requires extension of a triple helix across a bend at the approximate middle of S2 between the so-called heptad repeat 1 (HR1) and central helix (CH) domains.

As is common for integral membrane proteins, transport of the Spike polypeptide to the endoplasmic reticulum (ER) is initially driven by an N-terminal signal sequence of approximately twenty amino acids which is cleaved before release from the ER. During virus assembly in infected cells, transport of Spike through the Golgi apparatus to the cell membrane surface is restricted by nineteen amino acids localized to the intracellular C-terminal domain which function to insure co-assembly with other CoV structural proteins.

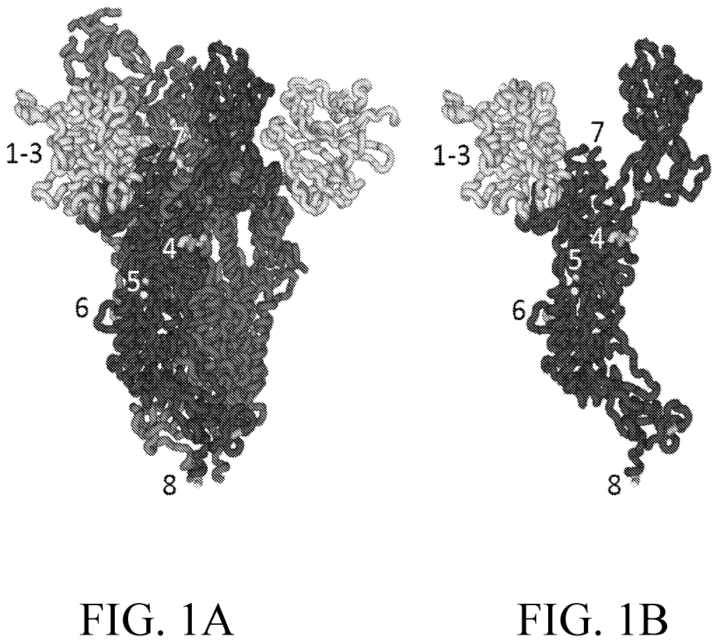

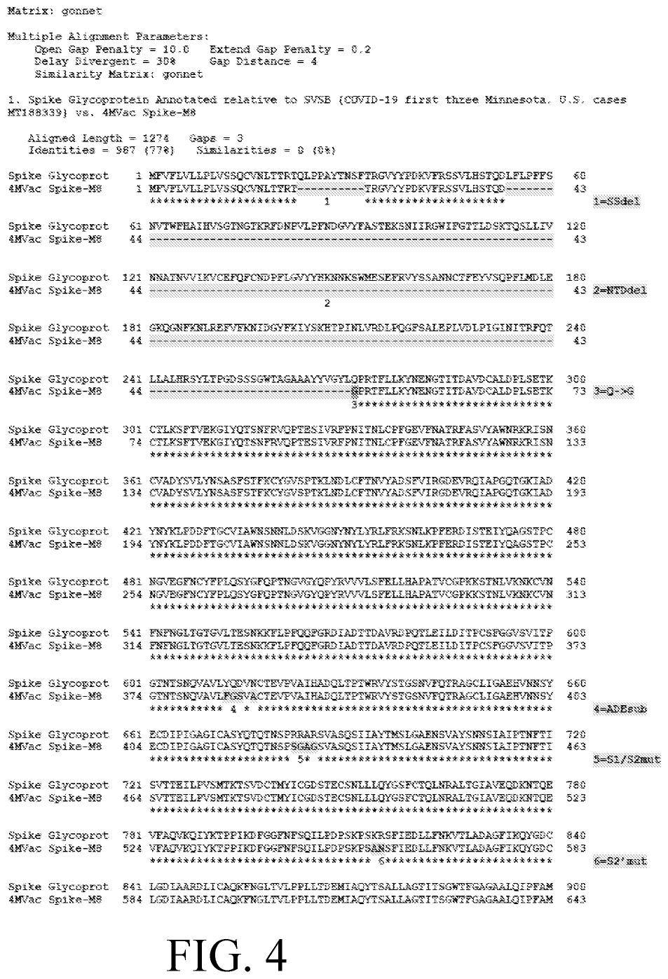

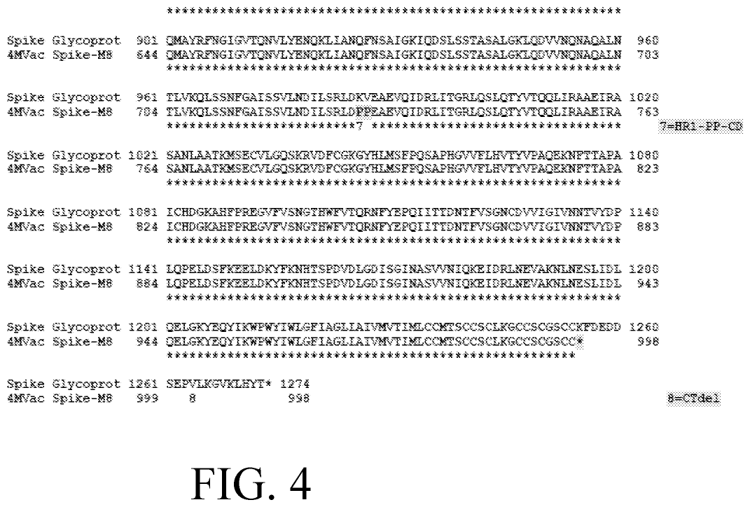

The biological basis for eight mutations that are introduced in various combinations in cis into the SARS CoV-2 Spike transgene, wherein the protein product with all eight mutations is designated CoV-2 Spike-M8 (also referred to as “SARS CoV-2 Spike-M8”, “4MVac Spike-M8” or “Spike-M8”) is described herein. The eight mutations are shown in FIG. 4. The relative positions of the eight mutations on the Spike protein are also shown in FIGS. 1A-1B.

The eight mutations are listed below with a description of the rationale used to arrive at the mutation.

1) Deletion of codons for 10-19 amino acids immediately 3′ of the coding sequence for the >˜13 amino acid sequence. This mutation is designed to eliminate the N-terminal-most portion of the N-terminal domain (NTD) so as to minimize ADE risk and to optimize the stability of folding.

2) Deletion further 3′ from 1), also in the NTD. This mutation is designed to eliminate potential ADE epitopes from a larger portion of the NTD while preserving a portion of a beta-pleated sheet that three-dimensionally abuts the receptor binding domain (RBD) in the Spike “crown.” A secondary rationale for this deletion is to shorten the coding region to enable the use of the full length cytomegalovirus (CMV) promoter/enhancer in the context of the recombinant adeno-associated virus (AAV) vectors. Recombinant AAVs have an empirically determined maximum packaging capacity of ˜5 kb, including the inverted terminal repeats (ITRs).

3) Substitution of Q(271)G at the C-terminus of deletion 2). This mutation is designed to improve the peptide chain flexibility and optimize the stability of protein folding.

4) Substitution of LYQDVN (SEQ ID NO: 7)(611-616)LFGSVA (SEQ ID NO: 3). This mutation is designed to alter four of the six amino acids in this region and thereby eliminate the ADE epitope. The substituting amino acids are based on VAST alignment to structures for other coronavirus Spike proteins.

5) Substitution of RRAR (SEQ ID NO: 8)(682-685)SGAG (SEQ ID NO: 6). This mutation is designed to eliminate the furin protease site at the S1/S2 junction.

6) Substitution of KR(814-815)AN. This mutation is designed to eliminate the protease site at S2′, immediately 5′ of the coding sequence for the fusion peptide.

7) Substitution of KV(986-987)PP at the HR1/CH junction. This mutation is designed to destabilize re-folding of the pre-fusion triple helix-loop-helix to the long triple helix characteristic of the post-fusion structures.

8) Deletion of KFDEDDSEPVLKGVKLHYT (SEQ ID NO: 9)(1255-1273), nineteen amino acids from the C-terminus. This mutation is designed to disrupt the binding of Spike to other CoV-2 structural proteins in the ER, thereby promoting transport of the Spike homotrimer to the cell membrane. Lontok et al. (Lontok et al., Journal of Virology, June 2004, Vol. 78, No. 11, p. 5913-5922) showed that the C-terminus of the full length SARS CoV coronavirus spike protein contained an endoplasmic reticulum retention signal. Mutation #8 should enable protein transport to the cell membrane with accelerated/enhanced presentation of the antigen.

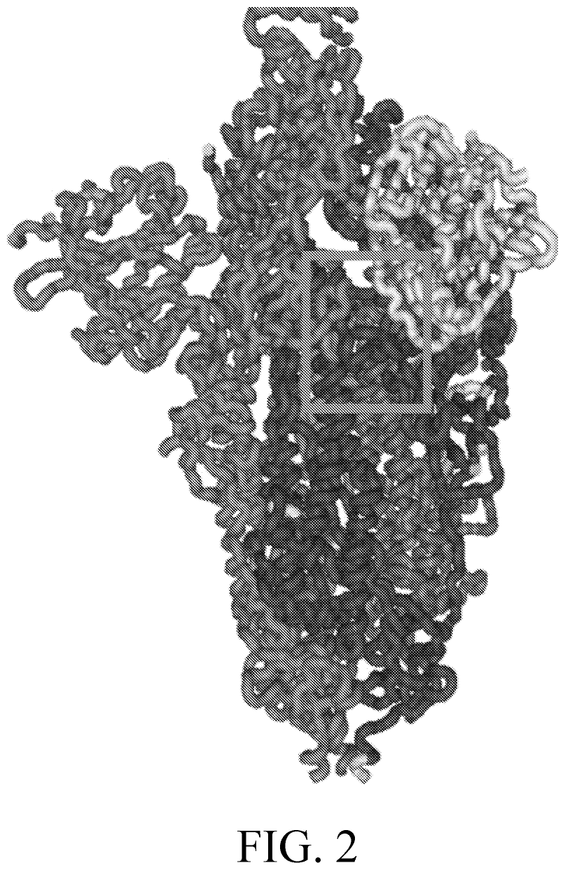

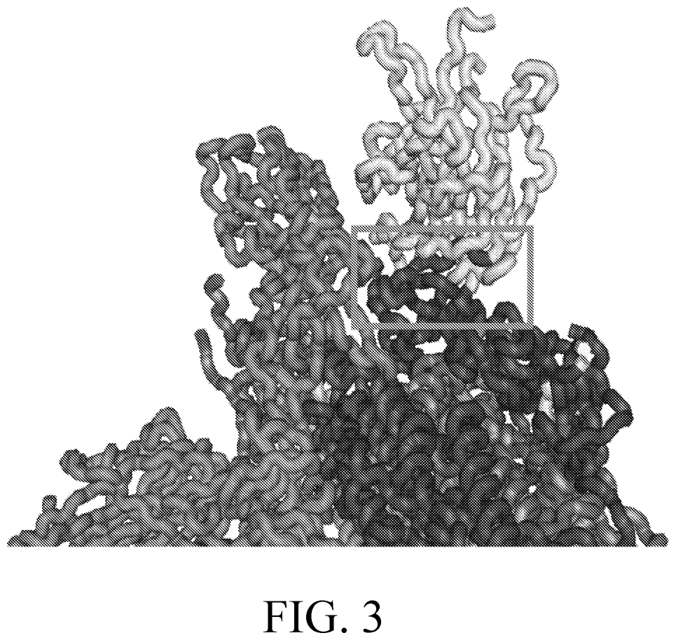

As shown in FIG. 2, the preservation of the interface between the protomers optimizes the chaperone function of the crown during subsequent folding of the portion corresponding to the S2 subfragment. FIG. 3 shows the interface between retained portions of one protomer against the other protomer, and the relative isolation of the deleted NTD from the rest of the protomer, as depicted with different shading.

Accordingly, in one aspect, a mutated SARS-CoV-2 S glycoprotein (mutated S glycoprotein) is provided. In some embodiments of any one of the aspects herein, the mutated S glycoprotein comprises a SARS-CoV-2 S glycoprotein amino acid sequence having one or more mutations compared to a wildtype S glycoprotein. In certain embodiments, the mutated S glycoprotein has one or more, all, or any combination of the following activities or properties: reduces, minimizes, or eliminates antibody-dependent enhancement (ADE); reduces, minimizes, or eliminates vaccine-associated enhanced respiratory disease (VAERD); increases or maximizes production of neutralizing antibodies to SARS-CoV-2; increases a subject's immune response to SARS-CoV-2; and increased production and/or increased quality of the S glycoproteins produced from a host cell, e.g., due to reduced degradation of the protein, decreased misfolding, or increased protein stability.

In some embodiments, the mutated S glycoprotein comprises a sequence having at least 40%, at least 45%, at least 50%, at least 55%, at least 60%, at least 65%, at least 70%, at least 75%, at least 80%, at least 85%, at least 90%, or at least 95% identity to SEQ ID NO: 1. In certain embodiments, the mutated S glycoprotein comprises one or more, all, or any combination of the following mutations:
(i) a deletion of one or more amino acids from a region spanning amino acid position 14 to amino acid position 32 of a wildtype precursor S glycoprotein of SEQ ID NO: 1;
(ii) a deletion of one or more amino acids from within an N-terminal domain of a wildtype precursor S glycoprotein of SEQ ID NO: 1, wherein the deletion removes epitopes in the wildtype mature S glycoprotein that contribute to ADE or VAERD;
(iii) an amino acid substitution at amino acid position 271 in a wildtype precursor S glycoprotein of SEQ ID NO: 1;
(iv) one or more amino acid substitutions in a region spanning amino acid position 611 to amino acid position 616 of wildtype precursor S glycoprotein of SEQ ID NO: 1;
(v) one or more amino acid substitutions in a region spanning amino acid position 682 to amino acid position 685 of wildtype precursor S glycoprotein of SEQ ID NO: 1;
(vi) an amino acid substitution at amino acid position 814 and/or amino acid position 815 of wildtype precursor S glycoprotein of SEQ ID NO: 1;
(vii) an amino acid substitution at amino acid position 986 and/or amino acid position 987 of wildtype precursor S glycoprotein of SEQ ID NO: 1; and
(viii) a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell.

In some embodiments, the mutated S glycoprotein comprises a sequence having at least 40%, at least 45%, at least 50%, at least 55%, at least 60%, at least 65%, at least 70%, at least 75%, at least 80%, at least 85%, at least 90%, or at least 95% identity to SEQ ID NO: 1, and comprises one or more, all, or any combination of the following mutations:
(i) a deletion of one or more amino acids from a region spanning amino acid position 14 to amino acid position 32 of a wildtype precursor S glycoprotein of SEQ ID NO: 1 (“mutation (i)”);
(ii) a deletion of one or more amino acids from within an N-terminal domain of a wildtype precursor S glycoprotein of SEQ ID NO: 1, wherein the deletion removes epitopes in the wildtype mature S glycoprotein that contribute to ADE or VAERD (“mutation (ii)”);
(iii) an amino acid substitution at amino acid position 271 in a wildtype precursor S glycoprotein of SEQ ID NO: 1 (“mutation (iii)”);
(iv) one or more amino acid substitutions in a region spanning amino acid position 611 to amino acid position 616 of wildtype precursor S glycoprotein of SEQ ID NO: 1 (“mutation (iv)”);
(v) one or more amino acid substitutions in a region spanning amino acid position 682 to amino acid position 685 of wildtype precursor S glycoprotein of SEQ ID NO: 1 (“mutation (v)”); (vi) an amino acid substitution at amino acid position 814 and/or amino acid position 815 of wildtype precursor S glycoprotein of SEQ ID NO: 1 (“mutation (vi)”);
(vii) an amino acid substitution at amino acid position 986 and/or amino acid position 987 of wildtype precursor S glycoprotein of SEQ ID NO: 1 (“mutation (vii)”); and
(viii) a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell (“mutation (viii)”).

In some embodiments, the mutated S glycoprotein comprises all of mutations (i) to (viii). In some embodiments, the mutated S glycoprotein comprises a sequence having at least 40%, at least 45%, at least 50%, at least 55%, at least 60%, at least 65%, at least 70%, at least 75%, at least 80%, at least 85%, at least 90%, or at least 95% identity to SEQ ID NO: 1, and comprises all of mutations (i) to (viii).

In some embodiments, the mutated S glycoprotein comprises a signal sequence. In some embodiments, the signal sequence comprises 10, 11, 12, 13, 14, 15, 16, 17, 18, 19, 20, 21, 22, 23, 24, or 25 amino acid residues. In some embodiments, the signal sequence comprises the amino acid sequence MFVFLVLLPLVSS (SEQ ID NO: 4), or a sequence with at least 80%, at least 85%, at least 90%, at least 95% identity thereto. In some embodiments, the signal sequence comprises the amino acid sequence MFVFLVLLPLVSSQCVNLTTRT (SEQ ID NO: 5), or a sequence with at least 80%, at least 85%, at least 90%, at least 95% identity thereto.

In some embodiments, the mutated S glycoprotein comprises a deletion of one or more amino acids from a region spanning amino acid position 14 to amino acid position 32 of a wildtype precursor S glycoprotein of SEQ ID NO: 1 (“mutation (i)”). In certain embodiments, mutation (i) comprises a deletion of each of the amino acids in the region spanning amino acid position 14 to amino acid position 32 of a wildtype precursor S glycoprotein of SEQ ID NO: 1. In some other embodiments, mutation (i) comprises a deletion of each of the amino acids in the region spanning acid position 14 to amino acid position 32, amino acid position 15 to amino acid position 32, amino acid position 16 to amino acid position 32, amino acid position 17 to amino acid position 32, amino acid position 18 to amino acid position 32, amino acid position 19 to amino acid position 32, amino acid position 20 to amino acid position 32, amino acid position 21 to amino acid position 32, amino acid position 22 to amino acid position 32, or amino acid position 23 to amino acid position 32. In certain embodiments, mutation (i) comprises a deletion of each of the amino acids in the region spanning amino acid position 23 to amino acid position 32 of a wildtype precursor S glycoprotein of SEQ ID NO: 1.

In some embodiments, the mutated S glycoprotein comprises a deletion of one or more amino acids from within an N-terminal domain of a wildtype precursor S glycoprotein of SEQ ID NO: 1, wherein the deletion removes epitopes in the wildtype mature S glycoprotein that contribute to ADE or VAERD (“mutation (ii)”). In certain embodiments, mutation (ii) comprises deletion of one or more amino acids from a region spanning amino acid position 24 to amino acid position 270 of a wildtype precursor S glycoprotein of SEQ ID NO: 1. In still other embodiments, mutation (ii) comprises deletion of each of the amino acids from the region spanning amino acid position 24 to amino acid position 270 of a wildtype precursor S glycoprotein of SEQ ID NO: 1.

In some embodiments, the mutated S glycoprotein comprises an amino acid substitution at amino acid position 271 in a wildtype precursor S glycoprotein of SEQ ID NO: 1 (“mutation (iii)”). In some embodiments, the amino acid substitution at amino acid position 271 of wildtype precursor S glycoprotein of SEQ ID NO: 1 is selected from the group consisting of: Q271G, Q271A, Q271I, Q271L, Q271M, Q271F, Q271P, and Q271V. In some embodiments, the amino acid substitution at amino acid position 271 of SEQ ID NO:1 is Q271G. In some other embodiments, the amino acid Q at position 271 of SEQ ID NO:1 is substituted with an amino acid residue with similar properties as G, e.g., a nonpolar and/or neutral amino acid residue.

In some embodiments, the mutated S glycoprotein comprises one or more amino acid substitutions in a region spanning amino acid position 611 to amino acid position 616 of wildtype precursor S glycoprotein of SEQ ID NO: 1 (“mutation (iv)”). In certain embodiments, the mutated S glycoprotein comprises a substitution at amino acid positions 612, 613, 614, and 616 of SEQ ID NO: 1. In some embodiments, the mutated S glycoprotein comprises a contiguous amino acid sequence of LFGSVA (SEQ ID NO: 3) at a region corresponding to amino acid position 611 to amino acid position 616 of wildtype precursor S glycoprotein of SEQ ID NO: 1. In some other embodiments, the mutated S glycoprotein comprises a contiguous amino acid sequence of amino acid residues with similar properties (e.g., polarity, acidity/basicity, hydrophobicity, aromaticity) as the amino acid residues LFGSVA (SEQ ID NO: 3) at a region corresponding to amino acid position 611 to amino acid position 616 of SEQ ID NO: 1.

In some embodiments, the mutated S glycoprotein comprises one or more amino acid substitutions in a region spanning amino acid position 682 to amino acid position 685 of wildtype precursor S glycoprotein of SEQ ID NO: 1 (“mutation (v)”). In some embodiments, the mutated S glycoprotein comprises a contiguous sequence of SGAG (SEQ ID NO: 6) at a region corresponding to amino acid position 682 to amino acid position 685 of wildtype precursor S glycoprotein of SEQ ID NO: 1. In some other embodiments, the mutated S glycoprotein comprises a contiguous amino acid sequence of amino acid residues with similar properties (e.g., polarity, acidity/basicity, hydrophobicity, aromaticity) as the amino acid residues SGAG (SEQ ID NO: 6) at a region corresponding to amino acid position 682 to amino acid position 685 of SEQ ID NO: 1.

In some embodiments, the mutated S glycoprotein comprises an amino acid substitution at amino acid position 814 and/or amino acid position 815 of wildtype precursor S glycoprotein of SEQ ID NO: 1 (“mutation (vi)”). In some embodiments, the mutated S glycoprotein comprises a contiguous sequence of AN at a region corresponding to amino acid position 814 to amino acid position 815 of wildtype precursor S glycoprotein of SEQ ID NO: 1. In some other embodiments, the mutated S glycoprotein comprises a contiguous amino acid sequence of amino acid residues with similar properties (e.g., polarity, acidity/basicity, hydrophobicity, aromaticity) as the amino acid residues AN at a region corresponding to amino acid position 814 to amino acid position 815 of SEQ ID NO: 1.

In some embodiments, the mutated S glycoprotein comprises an amino acid substitution at amino acid position 986 and/or amino acid position 987 of wildtype precursor S glycoprotein of SEQ ID NO: 1 (“mutation (vii)”). In some embodiments, the mutated S glycoprotein comprises a contiguous sequence of PP at a region corresponding to amino acid position 986 to amino acid position 987 of wildtype precursor S glycoprotein of SEQ ID NO: 1. In some other embodiments, the mutated S glycoprotein comprises a contiguous amino acid sequence of amino acid residues with similar properties (e.g., polarity, acidity/basicity, hydrophobicity, aromaticity) as the amino acid residues PP at a region corresponding to amino acid position 986 to amino acid position 987 of SEQ ID NO: 1.

In some embodiments, the mutated S glycoprotein comprises a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell (“mutation (viii)”). In some embodiments, the mutated S glycoprotein comprises a deletion of each of the amino acids in a region extending from amino acid position 1255 to amino acid position 1273 of wildtype precursor S glycoprotein of SEQ ID NO: 1.

In still other embodiments, the mutated S glycoprotein comprises a sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 2. SEQ ID NO: 2 is the sequence “4MVac Spike-M8” shown in FIG. 4, also reproduced below:

MFVFLVLLPLVSSQCVNLTTRTTRGVYYPDKVFRSSVLHST
QDGPRTFLLKYNENGTITDAVDCALDPLSETKCTLKSFTV
EKGIYQTSNFRVQPTESIVRFPNITNLCPFGEVFNATRFA
SVYAWNRKRISNCVADYSVLYNSASFSTFKCYGVSPTKLN
DLCFTNVYADSFVIRGDEVRQIAPGQTGKIADYNYKLPDD
FTGCVIAWNSNNLDSKVGGNYNYLYRLFRKSNLKPFERDI
STEIYQAGSTPCNGVEGFNCYFPLQSYGFQPTNGVGYQPY
RVVVLSFELLHAPATVCGPKKSTNLVKNKCVNFNFNGLTG
TGVLTESNKKFLPFQQFGRDIADTTDAVRDPQTLEILDIT
PCSFGGVSVITPGTNTSNQVAVLFGSVACTEVPVAIHADQ
LTPTWRVYSTGSNVFQTRAGCLIGAEHVNNSYECDIPIGA
GICASYQTQTNSPSGAGSVASQSIIAYTMSLGAENSVAYS
NNSIAIPTNFTISVTTEILPVSMTKTSVDCTMYICGDSTE
CSNLLLQYGSFCTQLNRALTGIAVEQDKNTQEVFAQVKQI
YKTPPIKDFGGFNFSQILPDPSKPSANSFIEDLLFNKVTL
ADAGFIKQYGDCLGDIAARDLICAQKFNGLTVLPPLLTDE
MIAQYTSALLAGTITSGWTFGAGAALQIPFAMQMAYRFNG
IGVTQNVLYENQKLIANQFNSAIGKIQDSLSSTASALGKL
QDVVNQNAQALNTLVKQLSSNFGAISSVLNDILSRLDPPE
AEVQIDRLITGRLQSLQTYVTQQLIRAAEIRASANLAATK
MSECVLGQSKRVDFCGKGYHLMSFPQSAPHGVVFLHVTYV
PAQEKNFTTAPAICHDGKAHFPREGVFVSNGTHWFVTQRN
FYEPQIITTDNTFVSGNCDVVIGIVNNTVYDPLQPELDSF
KEELDKYFKNHTSPDVDLGDISGINASVVNIQKEIDRLNE
VAKNLNESLIDLQELGKYEQYIKWPWYIWLGFIAGLIAIV
MVTIMLCCMTSCCSCLKGCCSCGSCC

In some embodiments, the mutated S glycoprotein comprises a sequence having at least 90% identity to SEQ ID NO: 2. In some embodiments, the mutated S glycoprotein comprises a sequence having at least 95% identity to SEQ ID NO: 2. In certain embodiments, the mutated S glycoprotein comprises SEQ ID NO: 2. In certain embodiments, the mutated S glycoprotein consists of SEQ ID NO: 2, or a sequence having at least 80%, at least 85%, at least 90%, at least 91%, at least 92%, at least 93%, at least 94%, at least 95%, at least 96%, at least 97%, at least 98%, or at least 99% sequence identity thereto. In some embodiments, the mutated S glycoprotein comprises one or more substitutions to SEQ ID NO: 2, wherein the substitution is a conservative substitution (e.g., substitution of an amino acid residue with a residue having similar properties, such as similar polarity, acidity/basicity, hydrophobicity, and/or aromaticity).

In another aspect, an isolated nucleic acid encoding a mutated SARS-CoV-2 S glycoprotein (mutated S glycoprotein) is provided. In yet another aspect, a vector comprising a nucleic acid encoding a mutated S glycoprotein is provided. The mutated S glycoprotein can be any mutated S glycoprotein described herein.

In some embodiments, the vector further comprises a promoter operatively linked to the nucleic acid sequence encoding the mutated S glycoprotein. In certain embodiments, the promoter is a muscle-specific promoter. In certain embodiments, the muscle-specific promoter is selected from the group consisting of: skeletal β-actin, myosin light chain 2A, dystrophin, muscle creatine kinase, SPc-512, and synthetic muscle promoters. In still other embodiments, the promoter is selected from the group consisting of: CMV, RSV, SV40, β-actin, PGK, and EF1 promoters.

The vector can be a plasmid. The vector can be a viral vector, such as a lentivirus vector, herpes virus vector, adenovirus vector or adeno-associated virus (AAV) vector. In some embodiments, the viral vector is a lentivirus vector. In some embodiment, the viral vector is a herpes virus vector. In some other embodiments, the viral vector is an AAV vector.

In some embodiments, the AAV vector comprises an AAV serotype 6 (AAV6) capsid protein. In some other embodiments, the AAV vector comprises an AAV serotype 9 (AAV9) capsid protein. In still other embodiments, the AAV vector comprises an Anc80, Anc80Lib, Anc 81, Anc82, Anc83, Anc84, Anc110, Anc113, Anc126, Anc127, or another phylogenetically related AAV capsid protein. In various embodiments the AAV is selected from the group consisting of AAV4, AAV5, RH33.34 and RH32.33.

In certain embodiments, the protection against viral challenge can occur in the absence of vaccine-induced neutralizing antibodies, thereby demonstrating that neutralizing humoral immunity is not a prerequisite for protection against SARS-CoV-2 infection.

In other embodiments, in addition to providing immediate protection against viral replication in host cells, strong and durable T cell memory in recipients of an AAV-M8/M8B vaccine can be expected to accelerate affinity maturation of neutralizing antibodies specific for a mutant RBD in any SARS-CoV-2 variant to which the host is initially exposed. Findings with regard to similar T cell reactivity to peptides from the alpha variant illustrate an example of such preservation of effector and helper T cell responses.

In another embodiment, the theoretical advantage of the variant-agnostic approach for a vaccinated population is its potential to circumvent the downside of antigenic imprinting to RBD conformational epitopes specific to the original Wuhan SARS-CoV-2 Spike. Among individuals in an outbred human population, the identity of immunodominant T cell epitopes will vary widely across the Spike sequence on the basis of HLA polymorphism, in contrast to the focal concentration of conformational B cell epitopes in the RBD to which most individuals must target neutralizing antibodies. Thus, the strength and durability of vaccine-induced T cell immunity has important implications for the future evolution of the COVID-19 pandemic as herd immunity to the original variant escalates and selective pressure builds for SARS-CoV-2 variants capable of escaping neutralizing humoral immunity through mutations in the RBD.

### Pharmaceutical Compositions and Formulations

In another aspect, a pharmaceutical composition comprising a mutated S glycoprotein as described herein is provided. In some embodiments, the pharmaceutical composition further comprises an adjuvant. In still another aspect, a vaccine composition comprising a mutated S glycoprotein as described herein is provided. In some embodiments, the vaccine composition further comprises an adjuvant. In some embodiments, the adjuvant is a CpG adjuvant.

Such a pharmaceutical composition may be in a form suitable for administration to a subject (i.e. mammal), or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In one embodiment, the pharmaceutical compositions useful for practicing the method of the invention may comprise an adjuvant. Non-limiting examples of suitable are Freund's complete adjuvant, Freund's incomplete adjuvant, Quil A, Detox, ISCOMs, squalene, or a CpG adjuvant. The pharmaceutical composition or vaccine composition can comprise any one or more of the adjuvants described herein.

Pharmaceutical compositions that are useful in the methods of the invention may be suitably developed for inhalation, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

The composition of the invention may comprise a preservative from about 0.005% to 2.0% by total weight of the composition. The preservative is used to prevent spoilage in the case of exposure to contaminants in the environment.

### Administration/Dosing

The regimen of administration may affect what constitutes an effective amount. For example, the compositions of the invention may be administered to the subject (i.e. mammal) in a single dose, in several divided dosages, as well as staggered dosages may be administered daily or sequentially, or the dose may be continuously infused, or may be a bolus injection. Further, the dosages may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat the disease in the subject. An effective amount of the composition necessary to achieve the intended result will vary and will depend on factors such as the disease to be treated or prevented, the age, sex, weight, condition, general health and prior medical history of the subject being treated, and like factors well-known in the medical arts. In particular embodiments, it is especially advantageous to formulate the composition in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the composition and the heterologous protein to be expressed, and the particular therapeutic effect to be achieved.

### Routes of Administration

One skilled in the art will recognize that although more than one route can be used for administration, a particular route can provide a more immediate and more effective reaction than another route. Routes of administration of any of the compositions of the invention include inhalation, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, electroporation and topical administration.

### Kits

In some embodiments a kit is provided for treating, preventing, or ameliorating an a given disease, disorder or condition, or a symptom thereof, as described herein wherein the kit comprises: a) a composition as described herein; and optionally b) an additional agent or therapy as described herein. The kit can further include instructions or a label for using the kit to treat, prevent, or ameliorate the disease, disorder or condition. In yet other embodiments, the invention extends to kits assays for a given disease, disorder or condition, or a symptom thereof, as described herein. Such kits may, for example, contain the reagents from PCR or other nucleic acid hybridization technology (microarrays) or reagents for immunologically based detection techniques (e.g., ELISpot, ELISA).

### Methods of Treatment

In one aspect, a method of inducing at least partial immunity to a coronavirus in a subject is provided. In another aspect, a method of generating an immune response against a coronavirus in a subject is provided. In yet another aspect, a method of vaccinating a subject against a coronavirus is provided. In still another aspect, a method of treating and/or preventing a disease or disorder associated with a coronavirus is provided. In certain embodiments, the coronavirus is SARS-CoV-2.

In some embodiments, the disease or disorder associated with a coronavirus is a respiratory disease. In some embodiments, the disease or disorder associated with a coronavirus is COVID-19.

In some embodiments, the method comprises administering to the subject a therapeutically effective amount of a mutated S glycoprotein, a pharmaceutical composition comprising a mutated S glycoprotein, a vector comprising a nucleic acid encoding a mutated S glycoprotein, or a vaccine composition comprising a mutated S glycoprotein. The mutated S glycoprotein can be any mutated S glycoprotein described herein.

In some embodiments, the administering minimizes antibody-dependent enhancement (ADE). In some other embodiments, the administering minimizes vaccine-associated enhanced respiratory disease (VAERD). In still other embodiments, the administering results in at least partial immunity to the coronavirus due to humoral immunity to the coronavirus. In some embodiments, the administering results in T-cell mediated immunity to the coronavirus. In some other embodiments, the administering results in an increase in titer of antibodies that specifically bind to the mutated S glycoprotein in the subject. In certain embodiments, the antibodies are neutralizing antibodies. In some embodiments, the administering results in a decrease in the rate of infection of the coronavirus in the subject. In some embodiments, the method further comprises administering an adjuvant to the subject, e.g., a CpG adjuvant.

In certain embodiments, the subject is a mammal. In some embodiments, the subject is a human. In some embodiments, the subject has been identified as not having previously had a coronavirus infection. In some embodiments, prior to the administering step, the subject has been identified as not having a significant titer of antibodies that bind specifically to the S glycoprotein or fragment thereof. In some embodiments, the subject has been previously identified as having one or more medical conditions selected from the group consisting of: chronic lung disease, moderate asthma, severe asthma, heart conditions, diabetes, obesity, liver disease, chronic kidney disease, and a weakened or suppressed immune system. In some embodiments, the subject identified as having a weakened or suppressed immune system is a subject receiving a cancer treatment, a smoker, a subject who is a transplant recipient, a subject having HIV or AIDS, or a subject receiving a corticosteroid or any other immunosuppressant drug. In some embodiments, the subject identified as having a weakened or suppressed immune system is an elderly subject.

Pharmaceutical compositions or vaccine compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The administration of the compositions of the invention may be carried out in any convenient manner known to those of skill in the art. The compositions of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook et al., 2012, volumes 1-4, Cold Spring Harbor Laboratory Press, NY); “Oligonucleotide Synthesis: A Practical Approach” (M. J. Gait, ed., Oxford University Press, 1984); “Culture of Animal Cells: A Manual of Basic Technique and Specialized Applications”, sixth edition (R. I. Freshney, Wiley-Blackwell, 2010); “Methods in Enzymology” (S. P. Colowick, N. O. Kaplan, et al., eds., volumes 1-650, Academic Press) “Handbook of Experimental Immunology”, fifth edition (D. M. Weir et al., Wiley, 1997); “Gene Transfer Vectors for Mammalian Cells” (J. Miller and M. P. Calos, Cold Spring Harbor Laboratory Press, N Y, 1987); “Short Protocols in Molecular Biology”, fifth edition (F. M. Ausubel et al., eds., John Wiley & Sons, 2002); “Polymerase Chain Reaction: Principles, Applications and Troubleshooting”, (M. E. Babar, VDM Verlag Dr. Muller, 2011); “Current Protocols in Immunology”, J. E. Coligan et al., eds., John Wiley & Sons, 1991). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the disclosure. The appended claims are intended to be construed to include all such embodiments and equivalent variations.

### Materials and Methods Utilized in the Following Examples

### M8/M8B Transgene Synthesis

De novo gene synthesis of M8 and MSB cDNA was performed by GeneArt using the codon optimization scheme from (Sino Biological, VG40589-UT). M8/M8B were cloned into the AAV transfer plasmid pZac2.1 along with (or flanked by) a CMV promoter and SV40 PolyA sequence. Cloned plasmids were transformed into Stbl2 competent cells and selected by a apicillin resistance.

### Immunocytochemistry of Transfected C2C12 Cells

3.0E4 C2C12 cells were plated into each well of an 8 well chamber slide (Millipore, PEZGS0816) and cultured for 24 hours in growth media (DMEM high glucose supplemented with 10% FBS, 1× anti-anti, 1× glutamax, 1×NEAA). Cells were then transfected with 0.4 ug plasmid DNA using lipofectamine 3000 in growth media for 5 hours at 37° C. before being switched to C2C12 differentiation medium (DMDM high glucose, 5% horse serum, 1× anti-anti, 1×NEAA, 1× glutamax) for 72 hours. Cells were then fixed with 10% NBF for 10 min at room temperature and then washed for 5 minutes with PBS before being permeabilized in 0.5% Triton X-100 solution 10 minutes at room temp. Cells were washed in PBS for 5 min before being blocked with 10% normal donkey serum (Abcam, ab7475, Lot GR3234297-32) diluted in PBS for 20 minutes at room temperature, followed by incubation with rabbit polyclonal a-SARS-CoV-2 Spike/RBD (Sino 40592-T62, Lot HD14AU0606 diluted 1:50 in PBS) for 1 hour at 37° C. Slides were then washed 3×10 minutes in PBS at RT and again blocked with 10% normal donkey serum (Abcam, ab7475, Lot GR3234297-32) for 20 minutes at room temperature before being incubated with PBS containing both WGA-555 (Thermo Fisher, W32464, diluted 1:40) and donkey a-rabbit-AF488 (Abcam, ab150061, diluted 1:500) for 30 minutes at 37° C. Following another 3×10 min washes in PBS, slides were mounted in VECTASHIELD mounting media containing DAPI (Vector Laboratories, H-1500). Images were taken using a Zeiss Observer 7 widefield microscope (Zeiss).

### Western Blot of Transfected HEK293

Cell pellets were lysed in RIPA lysis buffer (Santa Cruz Biotechnology, sc-24948) supplemented with protease inhibitor cocktail (Roche, 39802300). After homogenization, samples were spun at max speed for 15 minutes at 4° C. and the supernatant was then transferred to a new 1.5 ml microcentrifuge tube. Protein concentration was determined for each sample using the Pierce BCA protein assay kit (Thermo Scientific, 23227). Samples were then diluted to an appropriate concentration with 6× Laemmli SDS sample buffer (Alfa Aesar, J61337) before heat denaturing for 5 minutes at 95° C. 30 pg total protein was loaded per lane into and after running total protein was imaged for loading control using the REVERT Total Protein Stain Kit (LI-COR, 926-11010) and imaged by 700 nm fluorescence using the Odyssey Fc infrared imaging system (LI-COR). The membrane was then stained overnight at 4° C. in PBST+5% BSA containing anti-SARS-CoV-2 Spike/RBD (Sino 40592-T62, Lot HD14AU0606 diluted 1:1500). The membrane was then washed 3×15 minutes in PBST and then incubated in secondary antibody solution (PBST+5% BSA+Goat a-Rabbit-HRP 1:5000) (Abcam, ab205718) for one hour at room temperature. The membrane was then washed again 3×15 minutes in PBST before visualizing on the Odyssey Fc imaging system (LI-COR) using SuperSignal West Pico PLUS Chemiluminescent Substrate (Thermo Scientific, 34580).

### AAV6/9 Vector Generation

AAV6 and AAV9 were generated and purified by the University of Pennsylvania preclinical vector core using the triple transfection method in HEK293 cells as previously described [Song et al., Nature Medicine, 2019]. Vector preparations were assayed for quality, purity, and endotoxin levels before being pooled for injection. Vector aliquots were stored at −80° C. and thawed immediately prior to IM injection, except where explicitly stated for experimental purposes.

Vaccination of c57BL/10 and Kl8-hACE2 Mice

Immediately prior to IM injection, the AAV was removed from the −80° C. freezer and thawed at room temperature. The vector was then diluted in formulation buffer (PBS+0.001% Pluronic F-68) such that 50 ul of diluted vector contained the desired dose of viral genomes (either 6.4E10 or 1E12 vg), this controlled for volume injected across either dose administered. 2-5-month-old C57BL/10 mice or 6-9-week-old k18-hACE2 mice were anesthetized with isoflurane and received an IM injection of either 50 ul AAV or 50 ul formulation buffer into their left gastrocnemius using a custom 100 uL Hamilton syringe with a 32-gauge needle (475-41182, Lot 81008). Additionally, kl8-hACE2 mice were ear tagged as to keep the team performing the challenge experiments blinded to the identity of treatment status.

c57BL/10 Mouse Muscle Procurement, Sectioning, and Storage for Immunohistochemistry

At pre-determined timepoints post-vaccination, mice were anesthetized with 4% isoflurane in an anesthesia chamber before being transferred to a nose cone where they were maintained on 4% isoflurane in 100% 02 at a flow rate of 1 L/minute. Toe pinch was performed to ensure mice were deeply anesthetized before mice were euthanized by cervical dislocation. Both gastrocnemius muscles were harvested and embedded in O.C.T. (Tissue-Tek, 4583) within 7×7×5 mm base molds (Richard-Allan Scientific, 58949) by rapid submersion in liquid nitrogen-cooled isopentane. All OCT embedded tissue blocks were stored at −80° C. 12-24 hours before sectioning, OCT blocks were moved to −20° C. to gradually to warm to this temperature for optimal sectioning. Cryosections of 10-12 m thickness were cut on a cryostat (Microm HM550, Thermo Scientific) at −25° C. and mounted on glass slides (Fisher Scientific, Superfrost Plus, 12-550-15). Slides were allowed to air dry for 10 minutes at room temperature before being stored at −20° C.

Immunohistochemistry of c57BL/10 Gastrocnemius Muscle

10-12 m gastroenemius cross-section containing slides were removed from −20° C. and allowed to dry at room temperature for 10 minutes. Slides were then rehydrated for 10 minutes in PBS, permeabilized in 0.1% solution of Triton X-100 diluted in PBS for 10 minutes, and then washed again in PBS for 5 min. Sections were then blocked with 10% normal donkey serum (Abcam, ab7475, Lot GR3234297-32) diluted in PBS for 20 minutes at room temperature, followed by incubation with either rabbit polyclonal α-SARS-CoV-2 Spike/RBD (Sino 40592-T62, Lot HD14AU0606 diluted 1:50 in PBS) or goat anti-Utrophin for 1 hour at 37° C. Slides were then washed 3×10 minutes in PBS at RT and again blocked with 10% normal donkey serum (Abcam, ab7475, Lot GR3234297-32) for 20 minutes at room temperature before being incubated with PBS containing both WGA-555 (Thermo Fisher, W32464, diluted 1:40) and either donkey anti-rabbit-AF488 (Abcam, ab150061, diluted 1:500) or donkey anti-goat for 30 minutes at 37° C. Following another 3×10 min washes in PBS, slides were mounted in VECTASHIELD mounting media containing DAPI (Vector Laboratories, H-1500). Three 20×images were taken of each muscle section using a Zeiss Observer 7 widefield microscope (Zeiss).

### SARS-CoV-2 Viral Stock

SARS-CoV-2 strain USA-WA1/2020 was provided by BEI (NR-52281), propagation was performed by passaging two times in Vero E6 cells in DMEM supplemented with 2% fetal bovine serum and 1× Antibiotic Antimycotic Solution (sigma A5955). Supernatant was collected upon observation of cytopathic effect and centrifuged for debris removal. Titration was performed in triplicate by plaque assay in 6 well plates with crystal violet dye using a 200 μL inoculum per well from the generated stock. The produced stock with a final titer of 3×106 PFU/ml was aliquoted and stored at −80° C.

### SARS-CoV-2 Challenge

Infection of mice was conducted in a certified Animal Biosafety Level 3 (ABSL-3) laboratory at University of Wisconsin—Madison. The protocol for the challenge studies was approved by the University of Wisconsin Institutional Animal Care and Use Committee (Protocol Number V006324). Animals were anesthetized with 5% isoflurane, and 2.5×104 PFU of SARS-CoV-2 (USA-WA1/2020) in a 50 μL volume was administered via Intranasal inoculation using a micropipette tip. Animals were weighed and monitored for health issues, loss of weight, inappetence, activity reduction and breathing issues every day. Animals were euthanized by exposing them to an overdose of isoflurane in a closed chamber followed by cervical dislocation at day 7 post infection or when reached the human endpoint criteria (more than 20% of body weight lost, respiratory distress, inappetence, signs of dehydration, lack of mobility or critical body condition). Before euthanasia, blood serum was obtained by maxillary vein collection. After euthanasia, right lung for viral loads were collected, weighed, and homogenized in 1 mL of Trizol using a bead beater, after centrifugation, supernatant was stored at −80° C. Left lung was separated and inflated with 10% neutral buffered formalin using a 26G veterinary I.V. catheter and preserved in the formalin for histopathological changes evaluation.

### Treadmill Acclimatization and Physiological Assessment

Vaccinated and mock vaccinated kl8-hACE2 mice were acclimated to treadmill twice prior to assessing their 8-minute treadmill performance 6 days before SARS-CoV-2 infection. These same mice were then assessed according to the same performance test at 5 days post SARS-CoV-2 infection. Treadmill performance tests followed a sex specific, 8-minute speed ramping protocol and the distance recorded was either that maximally allowed by the protocol in 8-minutes or that which they had completed upon their fifth 1.2 mA electrical shock.

### Whole Body Plethysmography

A custom whole body plethysmography system was designed for use in the ABSL3 facility at the University of Wisconsin to minimize both the need for mouse handling and the potential for mouse-to-mouse SARS-CoV-2 contamination. Pressure deflections in 500 cc single-use chambers were recorded into Acqknowledge 4.2 files using the Biopac TSD160A, DA100C, UIM100A, ad MP150 hardware modules in series. The DA100C module was configured to the following settings: GAIN 1000, 10 HzLP ON, LP 5 kHz, and HP 0.05 Hz. Investigators blinded to group assignments recorded ear tag number of individual mice and then placed them into a chamber to enable a 60 second period of uninterrupted pressure recording. To minimize background noise, the raw signal collected was filtered via Acqknowledge 4.2 Software using the following settings: Low Pass sample rate at 8 Hz, High Pass sample rate at 1.5 Hz. Rate Signal parameter and identification of Peaks was determined using a threshold of 0.002 Volts. All files were then managed identically to enable extraction of average values and standard deviations for two parameters over 60 seconds: respiratory rate and amplitude, the latter proportional to relative tidal volume. The results for individual mice were normalized to baseline data obtained before inoculation with SARS-CoV-2. The sensitivity and specificity of the system was validated in mice recovering from inhalational general anesthesia using brief exposure to isoflurane at 4% of inspired gas.

### SARS-CoV-2 Viral Load Quantification

RNA was extracted from tissue homogenates using a Trizol/chloroform extraction method, and cDNA was subsequently synthesized using high-capacity cDNA to RNA-to-cDNA Kit (Thermo Scientific). SARS-CoV-2 RNA levels were measured by quantitative PCR using TaqMan Fast Universal PCR Master Mix (Thermo Fisher: Cat #4352042) on the Applied Biosystems 7500 Fast Dx Real-Time PCR System. SARS-CoV-2-specific primers and probe set from the CDC 2019-Novel Coronavirus (2019-nCoV) Real-Time RT-PCR Diagnostic Panel were used to target the SARS-CoV-2 nucleocapsid (IDT: Cat. #10006713). CoV_N1 F Primer: GACCCCAAAATCAGCGAAAT (SEQ ID NO: 10); CoV_N1 R primer: TCTGGTTACTGCCAGTTGAATCTG (SEQ ID NO: 11); CoV_N1 probe: FAM-ACCCCGCATTACGTTTGGTGGACC-BHQ1 (SEQ ID NO: 12). A standard curve was generated with SARS-CoV-2 nCov_N_Positive control plasmid (IDT: Cat. #10006625) to determine viral copy numbers. Viral gene expression levels were normalized to lung tissue weight.

### SARS-CoV-2 Neutralization Assay

Production of VSV pseudotyped with SARS-CoV-2 S: 293T cells plated 24 hours previously at 5×106 cells per 10 cm dish were transfected using calcium phosphate with 35 pg of pCGl SARS-CoV-2 S D614G deltalS expression plasmid encoding a codon optimized SARS-CoV2 S gene with an 18-residue truncation in the cytoplasmic tail (kindly provided by Stefan Pohlmann). Twelve hours post transfection the cells were fed with fresh media containing 5 mM sodium butyrate to increase expression of the transfected DNA. Thirty hours after transfection, the SARS-CoV-2 spike expressing cells were infected for 2-4 hours with VSV-G pseudotyped VSVAG-RFP at an MOI of ˜1-3. After infection, the cells were washed twice with media to remove unbound virus. Media containing the VSVAG-RFP SARS-CoV-2 pseudotypes was harvested 28-30 hours after infection and clarified by centrifugation twice at 6000 g then aliquoted and stored at −80° C. until used for antibody neutralization analysis. Antibody neutralization assay using VSVAG-RFP SARS-CoV-2: All sera were heat-inactivated for 30 minutes at 55° C. prior to use in neutralization assay. Vero E6 cells stably expressing 2.0). Th t serum-virus mixture was t t en used to replace the media on VeroE6 TMPRSS2 cells. 22 hours post infection, the cells were washed and fixed with 4% paraformaldehyde before visualization on an S6 FluoroSpot Analyzer (CTL, Shaker Heights OH). Individual infected foci were enumerated, and the values compared to control wells without antibody. The focus reduction neutralization titer 50% (FRNT50) was measured as the greatest serum dilution at which focus count was reduced by at least 50% relative to control cells that were infected with pseudotype virus in the absence of human serum. FRNT₅₀ titers for each sample were measured in at least two technical replicates and were reported for each sample as the geometric mean. TMPRSS2 were seeded in 100 pl at 2.5×10⁴ cells/well in a 96 well collagen coated plate. The next day, 2-fold serially diluted serum samples were mixed with VSVAG-RFP SARS-CoV-2 pseudotype virus (100-300 focus forming units/well) and incubated for 1 hr at 37° C. Also included in this mixture to neutralize any potential VSV-G carryover virus was 1E9F9, a mouse anti-VSV Indiana G, at a concentration of 600 ng/ml (Absolute AntiboOv, Ab014t2-

### Lung Histology

Left lung tissues were fixed in 10% neutral buffered formalin then paraffin-embedded and sectioned at 7 um. Sections were deparaffinized in two changes of xylene followed by rehydration through descending concentrations of ethanol (100%, 95%, 80%, water). Sections were stained in Gill's hematoxylin (Thermo Scientific Cat #72604) for 3 minutes, then rinsed under running tap water, differentiated in 0.1% acetic acid for 3 seconds, followed by a second rinse. Sections were then immersed in Scott's bluing reagent (Ricca Cat #6697-1) for 1 minute and transferred to water, then counterstained in eosin-Y solution (Thermo Fisher Cat #71204) for 30 seconds. Finally, slides were dehydrated from water through ascending concentration of ethanol (70%, 95%, two changes 100%), cleared in two changes of xylene, and mounted with Cytoseal 60 (Thermo Scientific Cat #8310-4) and coverslip. Sections were visualized by brightfield microscopy.

### ELISA

Flat bottom, high binding polystyrene 96-well plates (Corning, 9018) were coated overnight at 4° C. with either 2 ug/ml SARS-CoV-2 Spike Protein ECD (Sino Biological, 40589-V08B1) or 1 ug/ml SARS-CoV-2 Spike-RBD (Sino Biological, 40592-V08B-B) in PBS. The following day the coating buffer was aspirated, and the plate was washed with PBST and then dried. Once dried the plate was blocked with PBS+1% BSA for 2 hr at RT before heat-inactivated serum samples diluted in PBS+1% BSA were added for 1 hour at room temperature. The ELISA plate was then washed 4 times with PBS+0.05% Tween-20 (PBST), followed by addition of 100 uL of 1:2000 goat anti-mouse IgG-ALP (Abcam, ab97020) diluted in PBST+1% BSA. After 1 hour at room temperature, plates were washed 4 times with PBST and then developed with PNPP (Thermo, 37621) for 30 minutes at room temperature. Optical density (OD) was measured at 405 nm. Endpoint titers were calculated as the highest reciprocal dilution that emitted an optical density exceeding 3×secondary antibody only control.

### Mouse ELISpots

Spleens from mice were collected individually in RPMI1640 media supplemented with 10% FBS (R10) and penicillin/streptomycin and processed into single cell suspensions. Cell pellets were re-suspended in 5 mL of ACK lysis buffer (Life Technologies, Carlsbad, CA) for 5 min RT, and PBS was then added to stop the reaction. The samples were again centrifuged at 1500×g for 10 min, cell pellets re-suspended in R10, and then passed through a 45 m nylon filter before use in ELISpot assay. ELISpot assays were performed using the Mouse IFN-7, IL-2, IL-4, and IL-10 ELISpotPLUS plates (MABTECH). 96-well ELISpot plates precoated with capture antibody were blocked with R10 medium overnight at 4° C. 200,000 mouse splenocytes were plated into each well and stimulated for 20 h with pools of 15-mer peptides overlapping by nine amino acids from the SARS-CoV-2 Spike protein. Cells were stimulated with a final concentration of 5 μL of each peptide per well in RPMI+10% FBS (R10). The spots were developed based on manufacturer's instructions. R10 and cell stimulation cocktails (Invitrogen) were used for negative and positive controls, respectively. Spots were scanned and quantified by ImmunoSpot CTL reader. Spot number was calculated by subtracting the negative control wells.

### Macaques

All procedures were approved by the Institutional Animal Care and Use Committee of the Children's Hospital of Philadelphia (CHOP). Animals were negative for viral pathogens including SIV (simian immunodeficiency virus), STLV (simian-T-lymphotrophic virus), SRV (simian retrovirus), and B virus (herpesvirus 1) and housed in an AAALAC-accredited facility at CHOP on a 12-hour timed light/dark cycle. Animals received varied enrichment including food treats, manipulatives, visual and auditory stimuli, and social interactions throughout the study. Four 2-3 yo female cynomolgus monkeys (Macaco fascicularis) were treated with the clinical candidates intramuscularly at doses of 1E11 or 1E12 gc/animal. Serum and PBMC samples were obtained at baseline and 2-week intervals for analyses of immunogenicity. At necropsy, splenocytes, popliteal and inguinal lymph nodes and exposed and unexposed skeletal muscle were harvested for immunologic and histopathologic studies.

### Macaque Immunohistochemistry

Skeletal muscle harvested at necropsy was embedded in O.C.T. (Tissue-Tek, 4583) within 7×7×5 mm base molds (Richard-Allan Scientific, 58949) by rapid submersion in liquid nitrogen-cooled isopentane. All steps with tissue sectioning and immunohistochemistry (IHC) are the same as with mouse tissue through blocking in 10% normal donkey serum (Abeam, ab7475) for 20 minutes at RT. After blocking, slides were incubated in PBS with rat anti-CD8 (Bio-Rad, YTC182.20) plus either rabbit polyclonal anti-SARS-CoV-2 Spike/RBD (Sino, 40592-T62) or rabbit polyclonal anti-CDllb (Abeam, ab52478) for 1 hour at 37° C. Slides were then washed 3×10 minutes in PBS at RT and again blocked with 10% normal donkey serum (Abeam, ab7475) for 20 minutes at room temperature before being incubated with PBS containing WGA-555 (Thermo Fisher, W32464), donkey anti-rabbit-AF488 (Abeam, ab150061), and donkey anti-rat-AF647 (Abeam, ab150155) for 30 minutes at 37° C. Following another 3×10 min washes in PBS, slides were mounted in VECTASHIELD mounting media containing DAPI (Vector Laboratories, H-1500). Images were taken using a Zeiss Observer 7 widefield microscope (Zeiss).

### Macaque ELISpot

Monkey IFNy ELISpotPRO plates (MABTECH, 3421M-2AST-2) were washed with PBS and blocked overnight at 4° C. in RPMI10+ media. ZES macaque PBMCs were plated in RPMI10+ media and stimulated for 24 hrs (37° C., 5% COZ) with peptide pools of 15-mers with 11 amino acid overlap covering either the SI or S2 fragment of WAI SARS-CoV-2 spike protein (JPT, PM-WCPV-S-1) at a final concentration of 5 ug/ml. PM A/Ionomycin was used as a positive control (eBioscience, 00-4970-03) and RPMI10+ media alone was used as a negative control for each animal. Plates were then incubated in ddFbO for 2 minutes and then washed 5× with PBS before incubating with ALP-conjugated anti-IFNy antibody at the manufacture's recommended concentration in PBS+1% BSA for 2 hr at RT. Following 5×washes in PBS NBT/BCIP chromagen substrate solution was added for 15 min to allow spots to form. Spots were quantified on a CTL ImmunoSpot S6 Core Analyzer.

### Example 1: Expression of SARS-CoV-2 Spike-M8

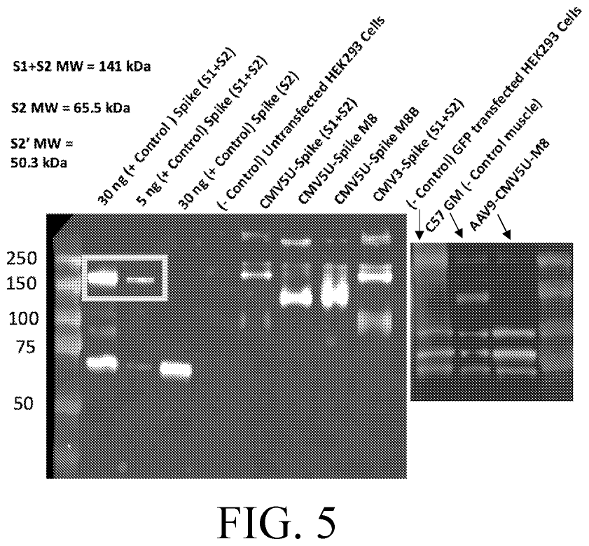

FIG. 5 shows robust expression of Spike-M8 in vitro and in vivo. HEK 293 cells were transfected with vectors encoding SARS-CoV-2 Spike-M8, vectors encoding a wildtype SARS-CoV-2 Spike protein as a positive control and vectors encoding GFP as a negative control. FIG. 5 (left) shows expression of SARS-CoV-2 Spike-M8 in HEK 293 cells, as shown by the band at ˜140 kDa in lanes labeled “CMV5U-Spike M8” and “CMV5U-Spike M8B.” The wildtype SARS-CoV-2 Spike protein in the lane labeled “CMV3-Spike (S1+S2)” appeared to have undergone proteolysis as indicated by the smeared bands. In contrast, the presence of a single bright band and absence of smeared bands in “CMV5U-M8” and “CMV5U-Spike M8B” shows that the SARS-CoV-2 Spike-M8 did not misfold or undergo proteolysis. Evidence is also provided for robust expression of Spike-M8 in vivo (mouse limb gastrocnemius muscle) at 7 days following intramuscular injection with an AAV vector encoding the Spike-M8 protein (FIG. 5, right).

### Example 2: Design and Characterization of AAV Vaccine

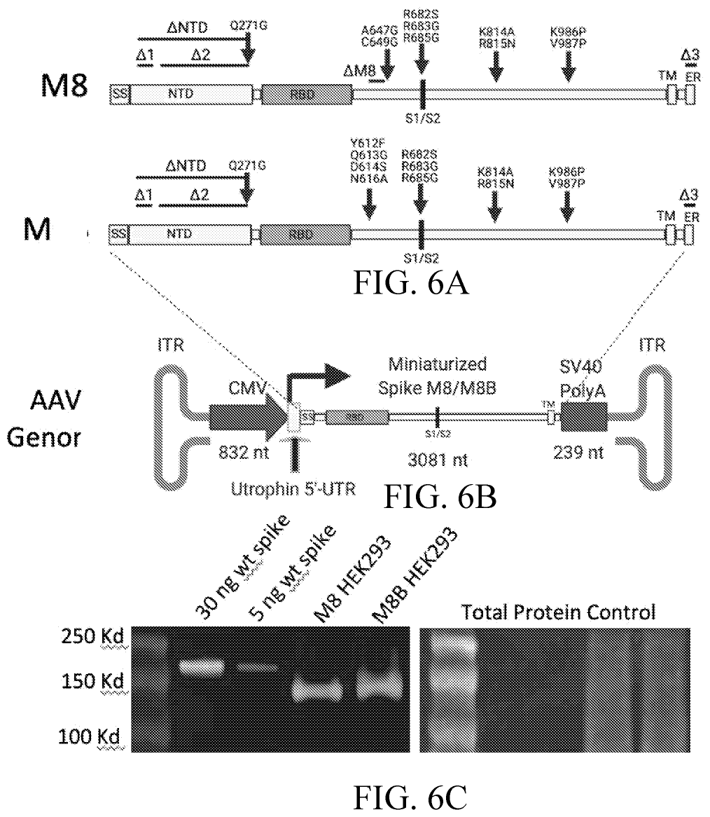

The original Wuhan strain (HB-01) spike protein is encoded by a 3,822-nucleotide open reading frame in the RNA genome [Zhou et al., Nature, 2020]. To accommodate the powerful and constitutive 832 bp CMV promoter used in our previous studies [Song et al., Nature Medicine, 2019], two AAV-deliverable transgenes encoding mutant spike derivatives were designed that selectively lack the autonomously folding N-terminal domain (ANTD) (FIG. 6A). Both transgenes contain 8 total mutational sites and are therefore designated “M8” and “M8B,” with 7 of the 8 mutations being identical between both transgenes. In addition to two N-terminal deletions (Δ1 and Δ2) that together constitute ANTD, both transgenes share Q271G to afford structural flexibility immediately following A2, mutated furin and S2′ proteolytic cleavage sites, proline mutations to stabilize prefusion structure, and deletion of the C-terminal ER-retention signal (A3) (FIG. 6A) [Mcallum et al., Nat. Struct., 2020] [Hoffann et al, Cell, 2020] [Pallesen et al., PNAS, 2017][McBride et al., JVI, 2007] [Ou et al., Nat. Comm. 2020] [Wrapp et al., Science, 2020]. The alternative 8th mutation was designed to selectively disrupt a conserved epitope encompassing the AA 614 position previously implicated in both antibody-dependent enhancement (ADE) in SARS-CoV and enhanced transmissibility of SARS-CoV-214-17 [Wang 2016 ACSID] [Yurkovetskiy 2020 Cell] [Hou 2020 Science] [Zhou 2021 Nature]. The packaged genomes consist of codon-optimized M8 and M8B open reading frames in an AAV ITR-flanked transcriptional cassette identical to that previously described 12 (FIG. 6B).

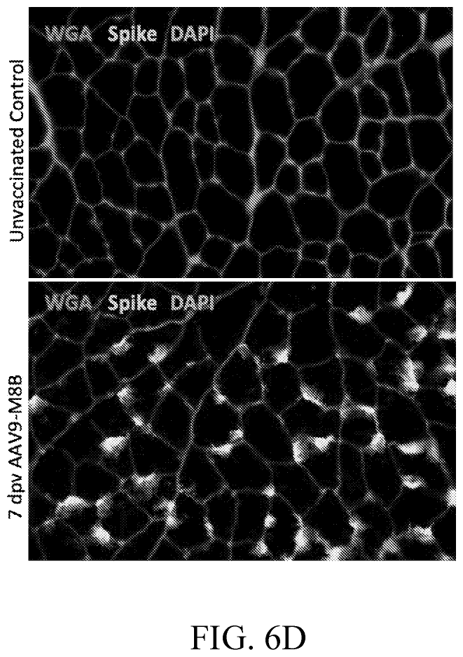

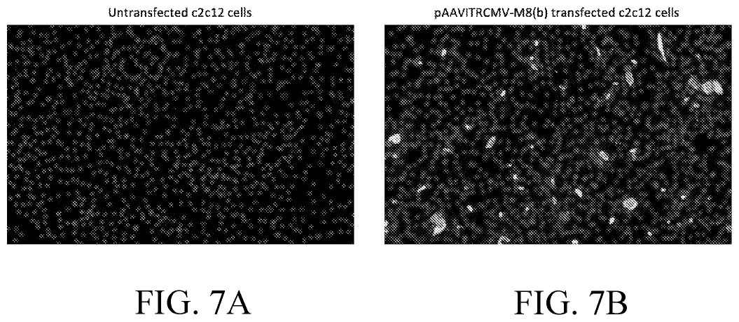

Robust expression of M8 and M8B was detected by western blot following transfection of HEK293 cells with plasmids containing the entire AAV-M8 and AAV-M8B genomes (FIG. 6C). M8B expression was additionally observed by immunofluorescent microscopy in transfected myogenic c2c12 cells (FIG. 7).

### Example 3: Vaccine-Induced Immunity in C57BL/10 Mice

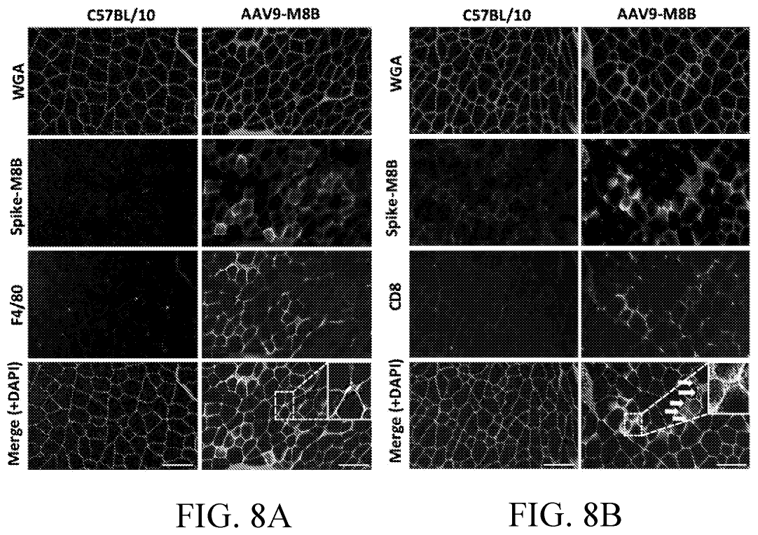

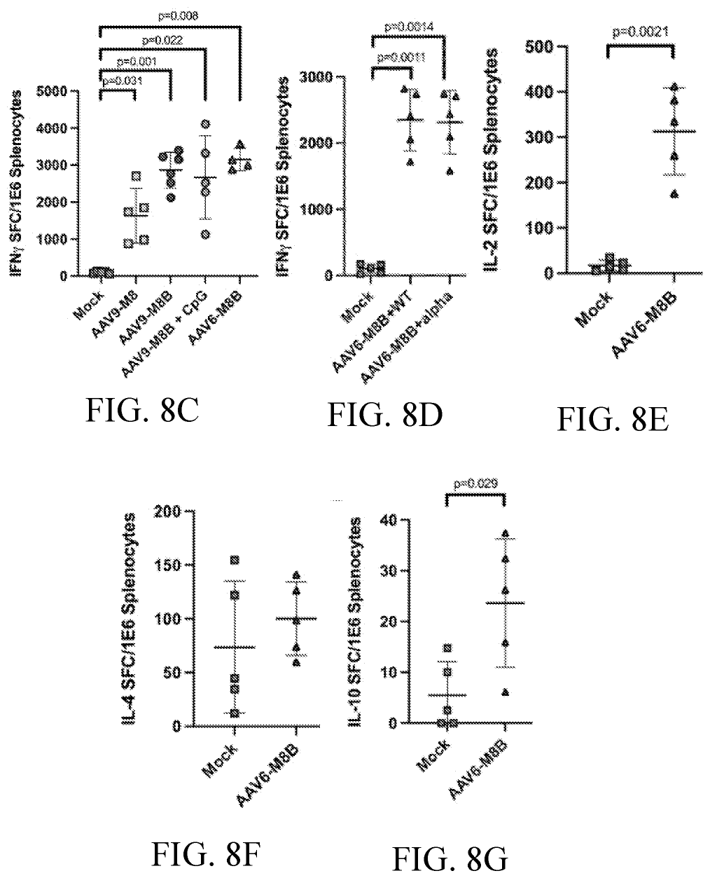

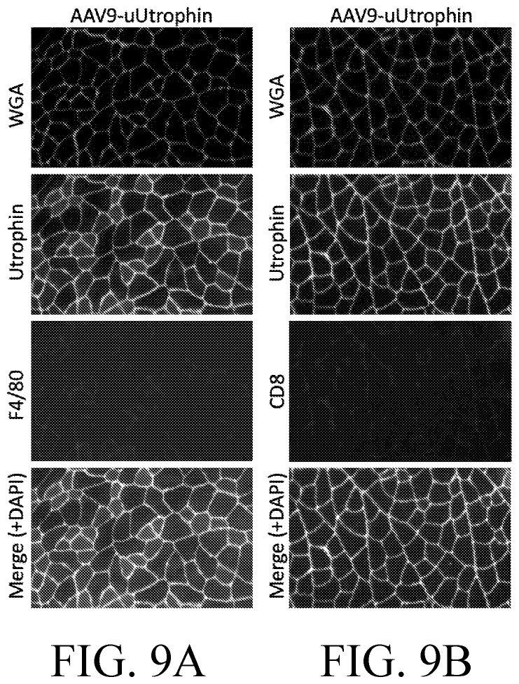

M8B was packaged into AAV9 (AAV9-M8B) and a group of eight C57BL/10 mice were administered 6.4E10 total viral genomes (vg) by an intramuscular (IM) route. Groups of two mice were euthanized at weekly intervals and immunohistochemistry was performed to assess the time course of M8B antigen expression as well as the overall muscle histology at the site of vaccination. At 7 days post vaccine administration (dpa) there was robust M8B expression within skeletal muscle fibers, while the muscle histology was otherwise normal (FIG. 6D). This finding remained unchanged until 21 dpa when regions of dense mononuclear infiltrates surrounded M8B-expressing muscle fibers. Immunohistological staining revealed F4/80+ macrophages (FIG. 8A) and CD8+ cytotoxic T cells (FIG. 8B) were present within the mononuclear infiltrates, indicating an ongoing cellular immune response targeting the transduced muscle fibers. Actively regenerating, centrally nucleated muscle fibers resembling those in murine muscular dystrophy are seen in close proximity to these infiltrating immune cells, further suggesting immune-mediated elimination of transduced muscle fibers12 (FIG. 8A, 8B, arrows). To control for immune response directed against the AAV9 vector capsid, C57BL/10 mice were injected with 6.4E10 vg AAV9-μUtrophin, a non-immunogenic vector in development for treatment of Duchenne muscular dystrophy12. At 21 dpa no macrophage or cytotoxic T cell infiltration was observed, despite intense focal expression of the non-immunogenic μUtrophin transgene product (FIG. 9).

To characterize the T cell responses, C57BL/10 mice received a single IM administration of either 6.4E10 vg AAV9-M8, AAV9-M8B, or AAV6-M8B and splenocytes were harvested one month post administration (mpa). Following ex vivo stimulation with peptide pools spanning the S1 and S2 fragment of spike, enzyme-linked immune absorbent spot (ELISpot) assays demonstrated a large number of INFγ-secreting cells in all vaccine groups (FIG. 8C), evidence of a strong T-helper-1 (Th1) response. These data indicate that a single IM dose of AAV-encoding either M8 or M8B is capable of eliciting a robust T cell mediated immune response against the AAV-encoded transgene product in vivo.

### Example 4: Immunity in and Protection of k18-hACE2 Mice

Angiotensin-converting enzyme 2 (ACE2) is the receptor for SARS-CoV-2 in humans [Zhou et al., Nature, 2020]. Wild type mice are not susceptible to SARS-CoV-2 infection because the spike RBD has low affinity to the mouse ACE2 ortholog. k18-hACE2 transgenic mice express human ACE2 (hACE2) driven by the cytokeratin 18 (kl8) promoter in airway and other epithelial cells [McCray Jr. et al., JVI, 2006]. It was recently demonstrated that inoculation of this strain with 10\hat{ }4 plaque-forming units (PFU) generates 50% lethal respiratory disease resembling severe COVID-19 [Winkler et al., Nature Immunology, 2020] [Zheng et al., Nature, 2020].

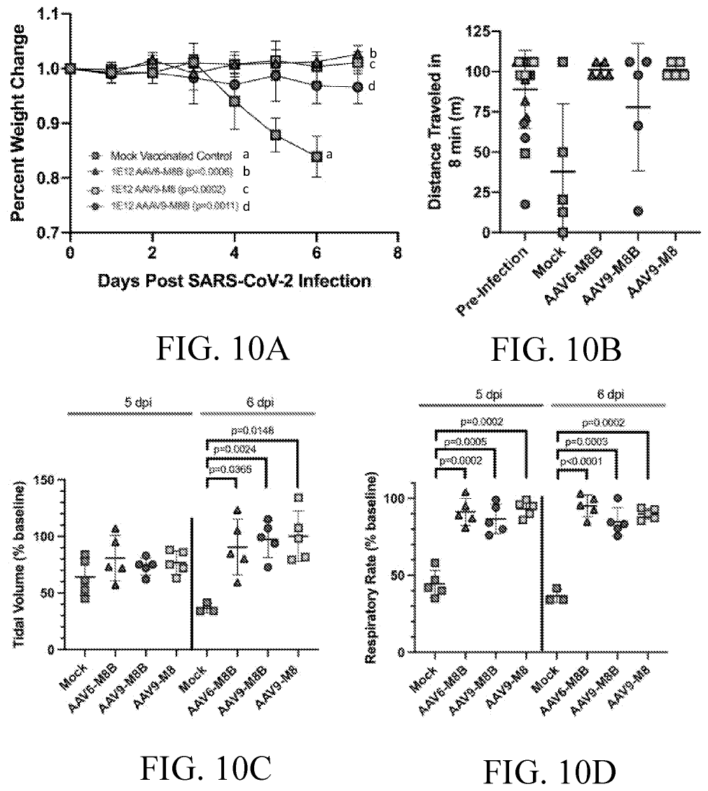

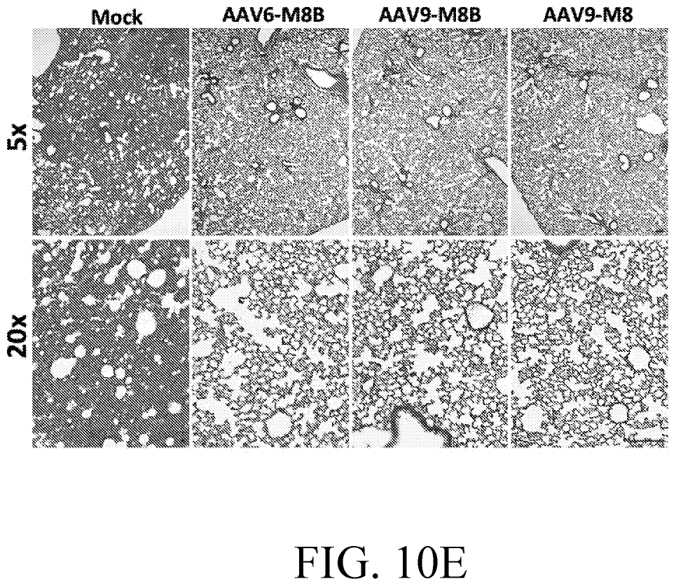

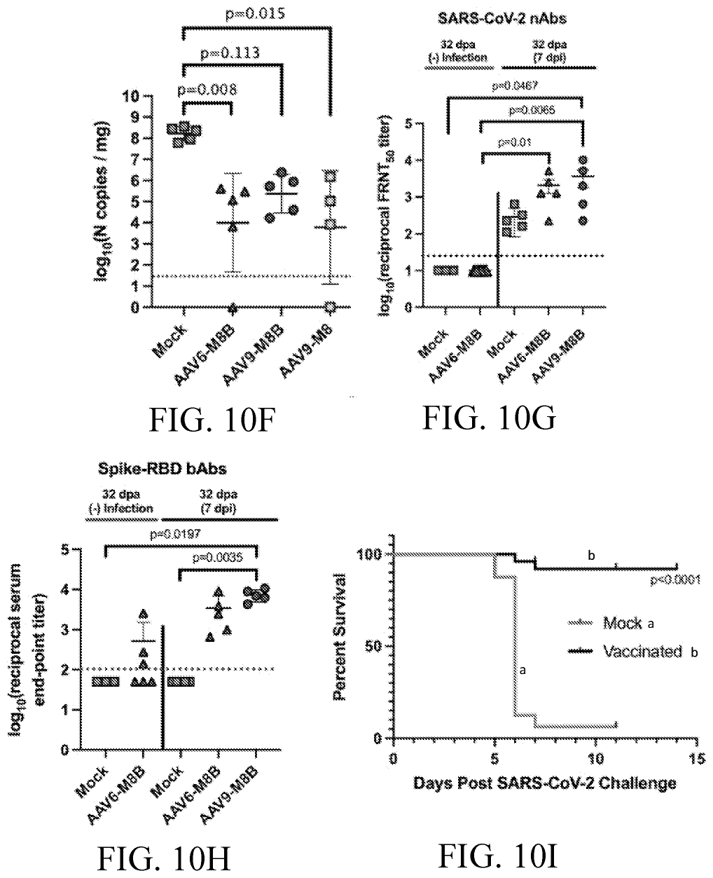

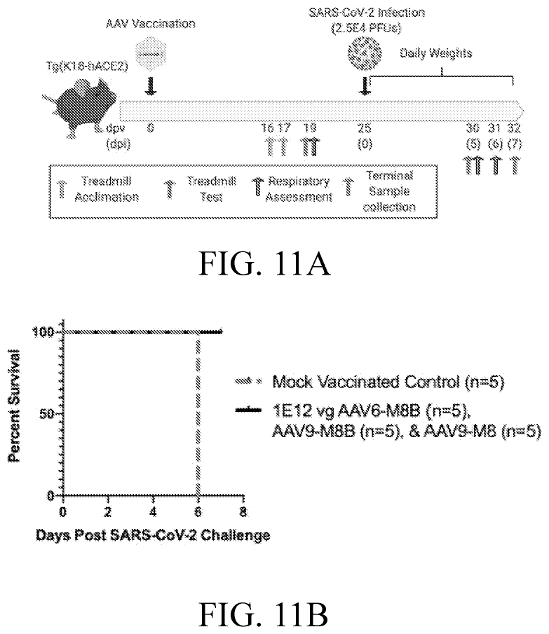

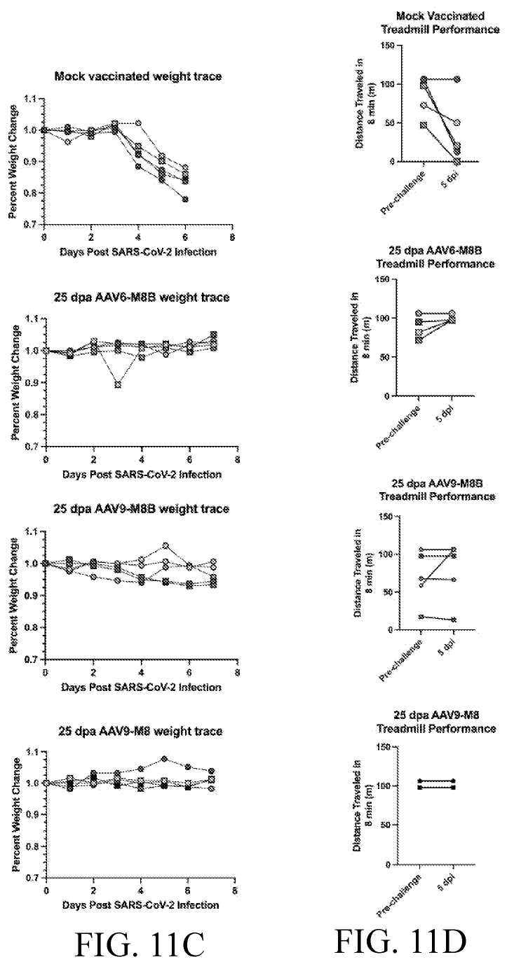

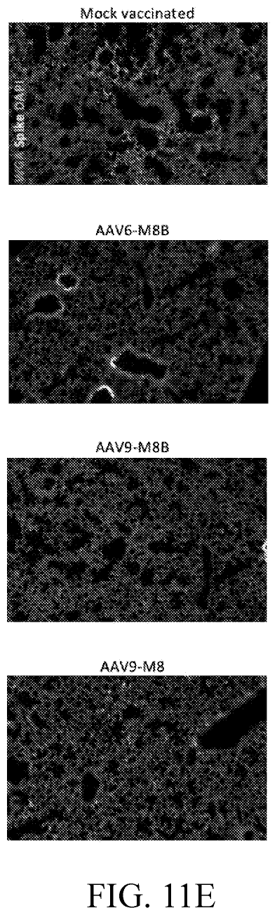

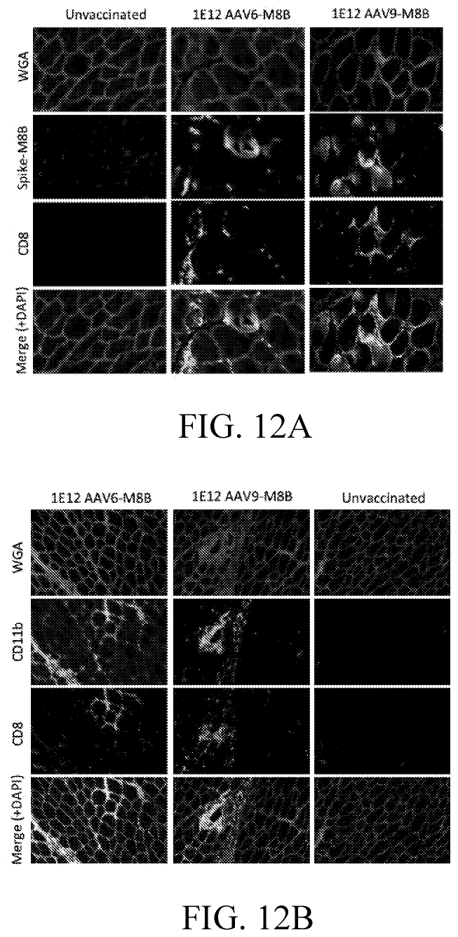

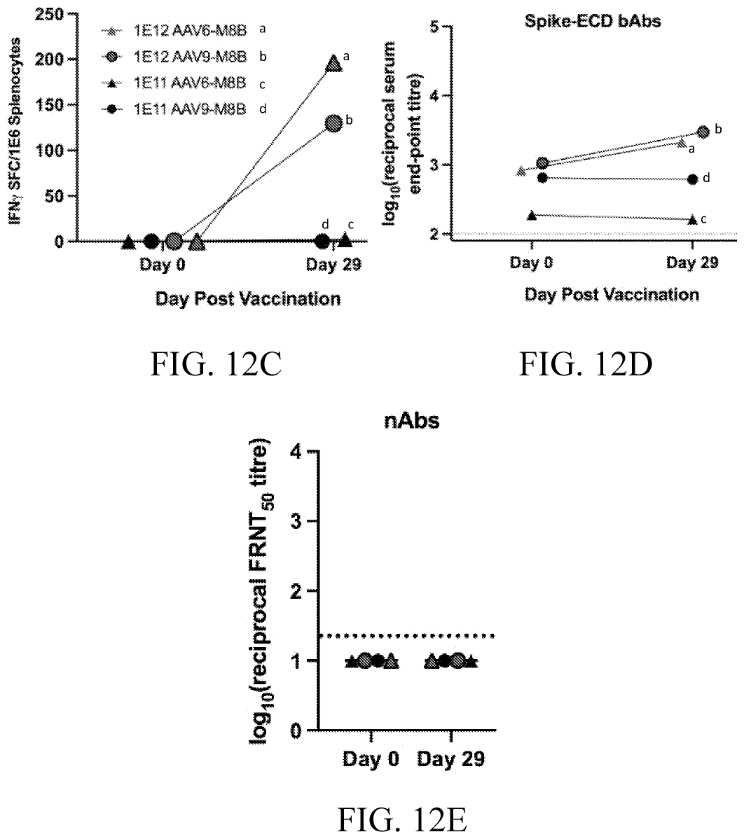

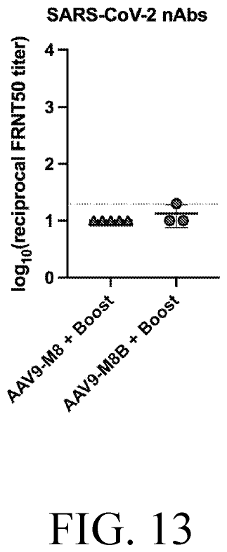

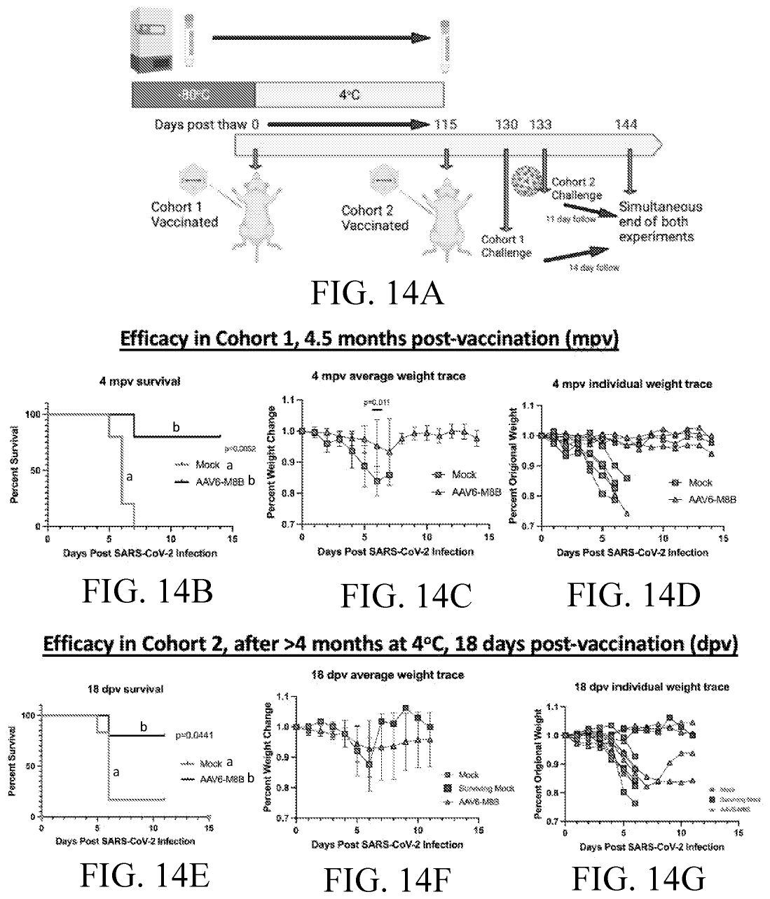

To characterize the vaccine-induced memory response in k18-hACE2 mice, animals received a single IM injection of either 1E12 vg AAV6-M8B or AAV buffer and splenocytes were harvested at 65 dpa. ELISpot revealed significant numbers of splenocytes secreted Th1-type cytokines INFγ and IL-2 upon ex vivo stimulation with peptide pools spanning the S1 and S2 fragment of spike (FIG. 8D, 8E). Interestingly, at 65 dpa there is no significant increase in number of splenocytes secreting the canonical T-helper-2 (Th2)-type cytokine IL-4 upon identical ex vivo stimulation (FIG. 8F); however, a significant, albeit very small total number of cells secrete IL-10 (FIG. 8G). Finally, to assess whether T cell responses are maintained in response to exposure to spike proteins from variant SARS-CoV-2 strains, a second INFγ ELISpot was run and stimulated ex vivo with peptide pools spanning the S1 and S2 fragments of spike protein of the alpha variant and there was no change in the number of INF Q secreting splenocytes (FIG. 8D). Together, these experiments indicate that the cellular memory response against the spike is strongly Th1-polarized, and this response is not diminished by mutations present in the alpha variant spike.
To assess vaccine efficacy against lethal SARS-CoV-2 challenge and associated viral pneumonia, 6-8-week-old k18-hACE2 mice were administered a single dose IM injection of either 1E12 vg AAV9-M8, AAV9-M8B, AAV6-M8B, or AAV buffer as a mock vaccinated control group before intranasal inoculation with a lethal dose of 2.5×10\hat{ }4 PFU of SARS-CoV-2 at 25 dpa (FIG. 11A). In order to compare tissues and viral RNA levels between groups, it was decided to terminate the experiment at 7 days post infection (dpi) with SARS-CoV-2, by which time the majority of kl8-hACE2 mice will have died or met objective criteria for euthanasia by the masked/blinded veterinarians overseeing the challenge experiments. As expected, by 6 dpi 100% of mock-vaccinated animals met terminal euthanasia criteria as determined by the presence of all of the following clinical findings: absence of movement, persistent eye closure with palpebral crusting, nasal secretions, kyphosis, and the presence of a rough coat resembling mice with the “rc” mutation [Cao 2007 JID]. No vaccinated animal demonstrated any of these clinical symptoms at any point in the 7-day experiment (FIG. 11B).
While the experimental design (termination at 7 dpi) did not allow for a longer assessment of differential survival, it did permit assessment of other objective surrogates of vaccine-induced protection. Mock-vaccinated mice began progressively losing weight starting at 4 dpi whereas animals in the vaccinated groups maintained stable weight over the 7 days post challenge (FIG. 10A, FIG. 11C). Animals were assessed for cardiopulmonary performance at 5 dpi by measuring the distance they were able to run on a treadmill for 8 minutes according to a predetermined ramping protocol. Whereas 80% of the mock vaccinated mice performed worse on the treadmill at 5 dpi than they did a week prior to viral challenge, all mice in each vaccinated group either improved or had no change in performance (FIG. 10B, FIG. 11D). Respiratory performance of these animals was assessed by whole body plethysmography at 5 and 6 dpi and there was a significant drop off in both tidal volume and respiratory rate in the mock vaccinated group at each of these time points relative to the pre-challenge assessment of all k18-hACE2 mice (FIG. 10C, 10D). Consistent with the observed differences in exercise tolerance and respiration, lungs of AAV-injected mice appeared grossly normal, while lungs of PBS-injected mice revealed signs of severe interstitial pneumonia characterized by collapse of the alveolar spaces (FIG. 10E). Viral RNA present in the lungs was quantified and a 2.85-log reduction for AAV9-M8B, a 4.44-log reduction for AAV9-M8, and a 4.3-log reduction for AA6-M8B vaccinated relative to the mock-vaccinated control group was observed (FIG. 10F), consistent with the significant decrease in spike protein in lung tissue sections by immunohistochemistry (FIG. 11E).
Interestingly, our analysis of serum obtained at necropsy revealed that this robust protection occurred despite the absence of vaccine-induced neutralizing humoral immunity (FIG. 10G). This finding was confirmed in mice by giving either AAV9-M8 or AAV9-M8B followed by a booster 5 weeks later. Three weeks post booster there was still an absence of detectable neutralizing antibody (FIG. 13). However, to support the claim AAV-M8B-primed CD4+ T cells enhance the kinetics of neutralizing antibody production post-SARS-CoV-2 infection, it was shown that neutralizing antibodies in the challenged AAV6-M8B and AAV9-M8B groups, but not the challenged mock vaccinated group, are significantly elevated relative to the unchallenged AAV6-M8B vaccinated group at the completion of the experiment (FIG. 10G). To further support the claim of accelerated post-vaccination, post-infection humoral immunity kinetics, IgG titers against recombinant spike RBD were assayed. Some of the unchallenged AAV6-M8B group did have detectable, albeit low levels, of anti-RBD IgG whereas all of the challenged mock vaccinated animals were below the limit of detection (FIG. 10H). However, the challenged AAV6-M8B and AAV9-M8B vaccinated mice were elevated 6.6-fold and 14.3-fold, respectively, relative to the unchallenged AAV6-M8B group and the challenged AAV9-M8B groups RBD IgG titer was significantly greater than that of the challenged mock vaccinated group (FIG. 10H). Together, these serological studies reveal that AAV-M8/M8B vaccination alone produces a very mild humoral immune response with no detectable neutralizing antibody component; however, upon infection vaccinated mice respond with an accelerated humoral response.
Next, long-term therapeutic efficacy was assessed as well as vaccine stability in real-world storage conditions, specifically following long-term refrigeration. A cryofrozen vial of AAV6-M8B was thawed and used to vaccinate k18-hACE2 mice (Cohort 1). The AAV6-M8B vial was then stored at 4° C. for 4 months before being used to vaccinate a second group of k18-hACE2 mice (Cohort 2) and then both cohorts were simultaneously challenged with 2.5×10\hat{ }4 PFU SARS-CoV-2 IN ˜18 days later (FIG. 14A). Long-term protection was observed from lethality and weight loss 4.5 months out from single dose vaccination in Cohort 1 (FIG. 14B-14D). Furthermore, long-term refrigeration did not impair its ability to protect against lethal challenge in Cohort 2 even at only 18 dpa (FIG. 14E); however, protection against weight loss was not statistically significant at this early timepoint post vaccination (FIG. 14F, 14G).
In aggregate, these challenge experiments demonstrate that vaccination with AAV-M8/M8B provides a highly significant survival advantage to kl8-hACE2 mice upon subsequent lethal challenge with SARS-CoV-2 (FIG. 10).

### Example 5: Vaccine-Induced Immunity in Macaques

To preliminarily assess capsid efficacy and dose-response in non-human primates, four cynomolgus macaques were vaccinated IM with either AAV6-M8B or AAV9-M8B at a dose of either 1E11 vg or 1E12 vg. At 029 dpa there is significant mononuclear cell infiltration and the presence of cytotoxic CD8 T cells and CD11b+ macrophages surrounding M8B expressing muscle fibers (FIG. 13A, 13B). INFγ ELISpots performed on peripheral blood mononuclear cells (PBMCs) isolated at 29 dpa demonstrated the presence of spike antigen-specific T cells in both macaques that received the higher dose of 1E12 vg, but neither animal that received the lower dose of 1E11 vg (FIG. 13C). Finally, as observed in mice, there was a small increase in the anti-Spike-ECD IgG titers of both macaques that received the 1E12 vg dose, but neither animal that received the dose of 1E11 vg and no vaccine-induced neutralizing antibodies were detectable at 29 dpa at any dose (FIG. 13D, 13E). These data strongly support our data generated from mice and suggest a similar immune response to AAV-M8B in primates.

### Enumerated Embodiments

The following enumerated embodiments are provided, the numbering of which is not to be construed as designating levels of importance.

Embodiment 1 provides a mutated SARS-CoV-2 S glycoprotein (mutated S glycoprotein) comprising a SARS-CoV-2 S glycoprotein amino acid sequence having one or more mutations compared to a wildtype S glycoprotein, wherein the mutated S glycoprotein minimizes (i) antibody-dependent enhancement (ADE) and/or (ii) vaccine-associated enhanced respiratory disease (VAERD) when administered to or expressed in a subject.

Embodiment 2 provides the mutated S glycoprotein of embodiment 1, wherein the mutated S glycoprotein comprises a sequence having at least 70% sequence identity to SEQ ID NO: 1 and one or more of:
(i) a deletion of one or more amino acids from a region spanning amino acid position 14 to amino acid position 32 of a wildtype precursor S glycoprotein of SEQ ID NO: 1;
(ii) a deletion of one or more amino acids from within an N-terminal domain of a wildtype precursor S glycoprotein of SEQ ID NO: 1, wherein the deletion removes epitopes in the wildtype mature S glycoprotein that contribute to ADE or VAERD;
(iii) an amino acid substitution at amino acid position 271 in a wildtype precursor S glycoprotein of SEQ ID NO: 1;
(iv) one or more amino acid substitutions in a region spanning amino acid position 611 to amino acid position 616 of wildtype precursor S glycoprotein of SEQ ID NO: 1;
(v) one or more amino acid substitutions in a region spanning amino acid position 682 to amino acid position 685 of wildtype precursor S glycoprotein of SEQ ID NO: 1;
(vi) an amino acid substitution at amino acid position 814 and/or amino acid position 815 of wildtype precursor S glycoprotein of SEQ ID NO: 1;
(vii) an amino acid substitution at amino acid position 986 and/or amino acid position 987 of wildtype precursor S glycoprotein of SEQ ID NO: 1; and
(viii) a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell.

Embodiment 3 provides the mutated S glycoprotein of embodiment 2, wherein the mutated S glycoprotein comprises a signal sequence.

Embodiment 4 provides the mutated S glycoprotein of any one of embodiments 2 and 3, wherein (i) comprises deletion of each of the amino acids in the region spanning amino acid position 14 to amino acid position 32 of a wildtype precursor S glycoprotein of SEQ ID NO: 1.

Embodiment 5 provides the mutated S glycoprotein of any one of embodiments 2-4, wherein (ii) comprises deletion of one or more amino acids from a region spanning amino acid position 24 to amino acid position 270 of a wildtype precursor S glycoprotein of SEQ ID NO: 1.

Embodiment 6 provides the mutated S glycoprotein of any one of embodiments 2-5, wherein (ii) comprises deletion of each of the amino acids from the region spanning amino acid position 24 to amino acid position 270 of a wildtype precursor S glycoprotein of SEQ ID NO: 1.

Embodiment 7 provides the mutated S glycoprotein of any one of embodiments 2-6, wherein the amino acid substitution at amino acid position 271 of wildtype precursor S glycoprotein of SEQ ID NO: 1 is selected from the group consisting of: Q271G, Q271A, Q271I, Q271L, Q271M, Q271F, Q271P, and Q271V.

Embodiment 8 provides the mutated S glycoprotein of any one of embodiments 2-7, wherein the mutated S glycoprotein comprises a contiguous amino acid sequence of LFGSVA (SEQ ID NO: 3) at a region corresponding to amino acid position 611 to amino acid position 616 of wildtype precursor S glycoprotein of SEQ ID NO: 1.

Embodiment 9 provides the mutated S glycoprotein of any one of embodiments 2-8, wherein the mutated S glycoprotein comprises a contiguous sequence of SGAG (SEQ ID NO: 6) at a region corresponding to amino acid position 682 to amino acid position 685 of wildtype precursor S glycoprotein of SEQ ID NO: 1.

Embodiment 10 provides the mutated S glycoprotein of any one of embodiments 2-9, wherein the mutated S glycoprotein comprises a contiguous sequence of AN at a region corresponding to amino acid position 814 to amino acid position 815 of wildtype precursor S glycoprotein of SEQ ID NO: 1.

Embodiment 11 provides the mutated S glycoprotein any one of embodiments 2-10, wherein the mutated S glycoprotein comprises a contiguous sequence of PP at a region corresponding to amino acid position 986 to amino acid position 987 of wildtype precursor S glycoprotein of SEQ ID NO: 1.

Embodiment 12 provides the mutated S glycoprotein of any one of embodiments 2-11, wherein the mutated S glycoprotein comprises a deletion of each of the amino acids in a region extending from amino acid position 1255 to amino acid position 1273 of wildtype precursor S glycoprotein of SEQ ID NO: 1.

Embodiment 13 provides the mutated S glycoprotein of any one of embodiments 1-12, wherein the mutated S glycoprotein comprises a sequence having at least 80% sequence identity to SEQ ID NO: 2.

Embodiment 14 provides a mutated SARS-CoV-2 S glycoprotein (mutated S glycoprotein) comprising a sequence that is at least 80% identical to SEQ ID NO: 2, wherein the mutated SARS-CoV-2 S glycoprotein minimizes (i) antibody-dependent enhancement (ADE) and/or (ii) vaccine-associated enhanced respiratory disease (VAERD) when administered to or expressed in a subject.

Embodiment 15 provides the mutated S glycoprotein of embodiment 14, wherein the mutated S glycoprotein comprises a sequence at least 90% identical to SEQ ID NO: 2.

Embodiment 16 provides the mutated S glycoprotein of embodiment 14, wherein the mutated S glycoprotein comprises a sequence at least 95% identical to SEQ ID NO: 2.

Embodiment 17 provides the mutated S glycoprotein of embodiment 16, wherein the mutated S glycoprotein comprises SEQ ID NO: 2.

Embodiment 18 provides a pharmaceutical composition comprising a mutated S glycoprotein of any one of embodiments 1-16.

Embodiment 19 provides the pharmaceutical composition of embodiment 17, further comprising an adjuvant.

Embodiment 20 provides a vaccine composition comprising a mutated S glycoprotein of any one of embodiments 1-16.

Embodiment 21 provides the vaccine composition of embodiment 19, further comprising an adjuvant.

Embodiment 22 provides a vector comprising a nucleic acid sequence encoding a mutated SARS-CoV-2 S glycoprotein (mutated S glycoprotein) comprising a SARS-CoV-2 S glycoprotein amino acid sequence having one or more mutations compared to a wildtype S glycoprotein, wherein the mutated S glycoprotein minimizes (i) antibody-dependent enhancement (ADE) and/or (ii) vaccine-associated enhanced respiratory disease (VAERD) when expressed in a subject.

Embodiment 23 provides the vector of embodiment 21, wherein the mutated S glycoprotein comprises a sequence having at least 70% sequence identity to SEQ ID NO: 1 and one or more of:
(i) a deletion of one or more amino acids from a region spanning amino acid position 14 to amino acid position 32 of a wildtype precursor S glycoprotein of SEQ ID NO: 1; (ii) a deletion of one or more amino acids from within an N-terminal domain of a wildtype mature S glycoprotein of SEQ ID NO: 2, wherein the deletion removes epitopes in the wildtype mature S glycoprotein that contribute to ADE or VAERD;
(iii) an amino acid substitution at amino acid position 271 in a wildtype precursor S glycoprotein of SEQ ID NO: 1;
(iv) one or more amino acid substitutions in a region spanning amino acid position 611 to amino acid position 616 of wildtype precursor S glycoprotein of SEQ ID NO: 1; (v) one or more amino acid substitutions in a region spanning amino acid position 682 to amino acid position 685 of wildtype precursor S glycoprotein of SEQ ID NO: 1;
(vi) an amino acid substitution at amino acid position 814 and/or amino acid position 815 of wildtype precursor S glycoprotein of SEQ ID NO: 1;
(vii) an amino acid substitution at amino acid position 986 and/or amino acid position 987 of wildtype precursor S glycoprotein of SEQ ID NO: 1; and
(viii) a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell.

Embodiment 24 provides the vector of embodiment 23, wherein the mutated S glycoprotein comprises a signal sequence.

Embodiment 25 provides the vector of embodiment 23 or 24, wherein (i) comprises deletion of each of the amino acids in the region spanning amino acid position 14 to amino acid position 32 of a wildtype mature S glycoprotein of SEQ ID NO: 1.

Embodiment 26 provides the vector of any one of embodiments 23-25, wherein (ii) comprises deletion of one or more amino acids from a region spanning amino acid position 24 to amino acid position 270 of a wildtype precursor S glycoprotein of SEQ ID NO: 1.

Embodiment 27 provides the vector of any one of embodiment 23-26, wherein (ii) comprises deletion of each of the amino acids from the region spanning amino acid position 24 to amino acid position 270 of a wildtype precursor S glycoprotein of SEQ ID NO: 1.

Embodiment 28 provides the vector of any one of embodiments 23-27, wherein the amino acid substitution at amino acid position 271 of wildtype precursor S glycoprotein of SEQ ID NO: 1 is selected from the group consisting of: Q271G, Q271A, Q271I, Q271L, Q271M, Q271F, Q271P, and Q271V.

Embodiment 29 provides the vector of any one of embodiments 23-28, wherein the mutated S glycoprotein comprises a contiguous amino acid sequence of LFGSVA (SEQ ID NO: 3) at a region corresponding to amino acid position 611 to amino acid position 616 of wildtype precursor S glycoprotein of SEQ ID NO: 1.

Embodiment 30 provides the vector of any one of embodiments 23-29, wherein the mutated S glycoprotein comprises a contiguous sequence of SGAG (SEQ ID NO: 6) at a region corresponding to amino acid position 682 to amino acid position 685 of wildtype precursor S glycoprotein of SEQ ID NO: 1.

Embodiment 31 provides the vector of any one of embodiments 23-30, wherein the mutated S glycoprotein comprises a contiguous sequence of AN at a region corresponding to amino acid position 814 to amino acid position 815 of wildtype precursor S glycoprotein of SEQ ID NO: 1.

Embodiment 32 provides the vector of any one of embodiments 23-31, wherein the mutated S glycoprotein comprises a contiguous sequence of PP at a region corresponding to amino acid position 986 to amino acid position 987 of wildtype precursor S glycoprotein of SEQ ID NO: 1.

Embodiment 33 provides the vector of any one of embodiments 23-32, wherein the mutated S glycoprotein comprises a deletion of each of the amino acids in a region extending from amino acid position 1255 to amino acid position 1273 of wildtype precursor S glycoprotein of SEQ ID NO: 1.

Embodiment 34 provides the vector of any one of embodiments 23-33, wherein the mutated S glycoprotein comprises a sequence having at least 80% sequence identity to SEQ ID NO: 2.

Embodiment 35 provides the vector of any one of embodiments 22-34 wherein the vector further comprises a promoter operatively linked to the nucleic acid sequence encoding the mutated S glycoprotein.

Embodiment 36 provides the vector of embodiment 35, wherein the promoter is a muscle-specific promoter.

Embodiment 37 provides the vector of embodiment 36, wherein the muscle-specific promoter is selected from the group consisting of: skeletal β-actin, myosin light chain 2A, dystrophin, muscle creatine kinase, SPc-512, and synthetic muscle promoters.

Embodiment 38 provides the vector of embodiment 35, wherein the promoter is selected from the group consisting of: CMV, RSV, SV40, β-actin, PGK, and EF1 promoters.

Embodiment 39 provides the vector of any one of embodiments 22-38, wherein the vector is a viral vector.

Embodiment 40 provides the vector of embodiment 39, wherein the viral vector is a lentivirus vector or herpes virus vector.

Embodiment 41 provides the vector of embodiment 39, wherein the vector is an AAV vector.

Embodiment 42 provides the vector of embodiment 41, wherein the AAV vector comprises an AAV serotype 6 (AAV6) capsid protein.

Embodiment 43 provides the vector of embodiment 41, wherein the AAV vector comprises an AAV serotype 9 (AAV9) capsid protein.

Embodiment 44 provides the vector of embodiment 41, wherein the AAV vector comprises an Anc80, Anc80Lib, Anc 81, Anc82, Anc83, Anc84, Anc110, Anc 13, Anc126, Anc127, or another phylogenetically related AAV capsid protein.

Embodiment 45 provides the vector of any one of embodiments 22-38, wherein the vector is a plasmid.

Embodiment 46 provides a pharmaceutical composition comprising a vector of any one of embodiments 22-45.

Embodiment 47 provides the pharmaceutical composition of embodiment 46, wherein the pharmaceutical composition further comprises an adjuvant.

Embodiment 48 provides the pharmaceutical composition of embodiment 47, wherein the adjuvant is a CpG adjuvant.

Embodiment 49 provides a vaccine composition comprising a vector of any one of embodiments 22-45.

Embodiment 50 provides the vaccine composition of embodiment 49, further comprising an adjuvant.

Embodiment 51 provides the vaccine composition of embodiment 50, wherein the adjuvant is a CpG adjuvant.

Embodiment 52 provides a method of inducing at least partial immunity to a coronavirus in a subject, the method comprising administering to the subject a therapeutically effective amount of a mutated S glycoprotein of any one of embodiments 1-17, a pharmaceutical composition of any one of embodiments 18, 19, and 46-48, a vector of any one of embodiments 22-45, or a vaccine composition of any one of embodiments 20, 21, and 49-51.

Embodiment 53 provides the method of embodiment 52, wherein the administering minimizes antibody-dependent enhancement (ADE).

Embodiment 54 provides the method of embodiment 52 or 53, wherein the administering minimizes vaccine-associated enhanced respiratory disease (VAERD).

Embodiment 55 provides the method of any one of embodiments 52-54, wherein the administering results in at least partial immunity to the coronavirus due to humoral immunity to the coronavirus.

Embodiment 56 provides the method of any one of embodiments 52-55, wherein the administering results in T-cell mediated immunity to the coronavirus.

Embodiment 57 provides the method of any one of embodiments 52-56, wherein the administering results in an increase in titer of antibodies that specifically bind to the mutated S glycoprotein in the subject.

Embodiment 58 provides the method of any one of embodiments 52-57, wherein the administering results in a decrease in the rate of infection of the coronavirus in the subject.

Embodiment 59 provides the method of any one of embodiments 52-58, wherein the method further comprises administering an adjuvant to the subject.

Embodiment 60 provides the method of embodiment 59, wherein the adjuvant is a CpG adjuvant.

Embodiment 61 provides the method of any one of embodiments 52-60, wherein the subject has been identified as not having previously had a coronavirus infection.

Embodiment 62 provides the method of any one of embodiments 52-61, wherein, prior to the administering step, the subject has been identified as not having a significant titer of antibodies that bind specifically to the S glycoprotein of the fragment thereof.

Embodiment 63 provides the method of any one of embodiments 52-62, wherein the coronavirus is SARS-CoV-2.

Embodiment 64 provides the method of any one of embodiments 52-63, wherein the subject has been previously identified as having one or more medical conditions selected from the group consisting of: chronic lung disease, moderate asthma, severe asthma, heart conditions, diabetes, obesity, liver disease, chronic kidney disease, and a weakened or suppressed immune system.

Embodiment 65 provides the method of embodiment 64, wherein the subject having a weakened or suppressed immune system is a subject receiving a cancer treatment, a smoker, a subject who is a transplant recipient, a subject having HIV or AIDS, or a subject receiving a corticosteroid or any other immunosuppressant drug.

Embodiment 66 provides the method of embodiment 64, wherein the subject having a weakened or suppressed immune system is an elderly subject.

1. A mutated SARS-CoV-2 S glycoprotein (mutated S glycoprotein) comprising a SARS-CoV-2 S glycoprotein amino acid sequence having one or more mutations compared to a wildtype S glycoprotein, wherein the mutated S glycoprotein minimizes (i) antibody-dependent enhancement (ADE) and/or (ii) vaccine-associated enhanced respiratory disease (VAERD) when administered to or expressed in a subject.

2. The mutated S glycoprotein of claim 1, wherein the mutated S glycoprotein comprises a sequence having at least 70% sequence identity to SEQ ID NO: 1 and one or more of:
(i) a deletion of one or more amino acids from a region spanning amino acid position 14 to amino acid position 32 of a wildtype precursor S glycoprotein of SEQ ID NO: 1;
(ii) a deletion of one or more amino acids from within an N-terminal domain of a wildtype precursor S glycoprotein of SEQ ID NO: 1, wherein the deletion removes epitopes in the wildtype mature S glycoprotein that contribute to ADE or VAERD;
(iii) an amino acid substitution at amino acid position 271 in a wildtype precursor S glycoprotein of SEQ ID NO: 1;
(iv) one or more amino acid substitutions in a region spanning amino acid position 611 to amino acid position 616 of wildtype precursor S glycoprotein of SEQ ID NO: 1;
(v) one or more amino acid substitutions in a region spanning amino acid position 682 to amino acid position 685 of wildtype precursor S glycoprotein of SEQ ID NO: 1;
(vi) an amino acid substitution at amino acid position 814 and/or amino acid position 815 of wildtype precursor S glycoprotein of SEQ ID NO: 1;
(vii) an amino acid substitution at amino acid position 986 and/or amino acid position 987 of wildtype precursor S glycoprotein of SEQ ID NO: 1; and
(viii) a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell.

3. The mutated S glycoprotein of claim 2, wherein the mutated S glycoprotein comprises a signal sequence.

4. The mutated S glycoprotein of claim 2, wherein (i) comprises deletion of each of the amino acids in the region spanning amino acid position 14 to amino acid position 32 of a wildtype precursor S glycoprotein of SEQ ID NO: 1.

5. The mutated S glycoprotein of claim 2, wherein (ii) comprises deletion of one or more amino acids from a region spanning amino acid position 24 to amino acid position 270 of a wildtype precursor S glycoprotein of SEQ ID NO: 1.

6. The mutated S glycoprotein of claim 2, wherein (ii) comprises deletion of each of the amino acids from the region spanning amino acid position 24 to amino acid position 270 of a wildtype precursor S glycoprotein of SEQ ID NO: 1.

7. The mutated S glycoprotein of claim 2, wherein the amino acid substitution at amino acid position 271 of wildtype precursor S glycoprotein of SEQ ID NO: 1 is selected from the group consisting of: Q271G, Q271A, Q271I, Q271L, Q271M, Q271F, Q271P, and Q271V.

8. The mutated S glycoprotein of claim 2, wherein the mutated S glycoprotein comprises a contiguous amino acid sequence of LFGSVA (SEQ ID NO: 3) at a region corresponding to amino acid position 611 to amino acid position 616 of wildtype precursor S glycoprotein of SEQ ID NO: 1.

9. The mutated S glycoprotein of claim 2, wherein the mutated S glycoprotein comprises a contiguous sequence of SGAG (SEQ ID NO: 6) at a region corresponding to amino acid position 682 to amino acid position 685 of wildtype precursor S glycoprotein of SEQ ID NO: 1.

10. The mutated S glycoprotein of claim 2, wherein the mutated S glycoprotein comprises a contiguous sequence of AN at a region corresponding to amino acid position 814 to amino acid position 815 of wildtype precursor S glycoprotein of SEQ ID NO: 1.

11. The mutated S glycoprotein any-ene of claim 2, wherein the mutated S glycoprotein comprises a contiguous sequence of PP at a region corresponding to amino acid position 986 to amino acid position 987 of wildtype precursor S glycoprotein of SEQ ID NO: 1.

12. The mutated S glycoprotein of claim 2, wherein the mutated S glycoprotein comprises a deletion of each of the amino acids in a region extending from amino acid position 1255 to amino acid position 1273 of wildtype precursor S glycoprotein of SEQ ID NO: 1.

13. The mutated S glycoprotein of claim 1, wherein the mutated S glycoprotein comprises a sequence having at least 80% sequence identity to SEQ ID NO: 2.

14. A mutated SARS-CoV-2 S glycoprotein (mutated S glycoprotein) comprising a sequence that is at least 80% identical to SEQ ID NO: 2, wherein the mutated SARS-CoV-2 S glycoprotein minimizes (i) antibody-dependent enhancement (ADE) and/or (ii) vaccine-associated enhanced respiratory disease (VAERD) when administered to or expressed in a subject.

15. The mutated S glycoprotein of claim 14, wherein the mutated S glycoprotein comprises a sequence at least 90% identical to SEQ ID NO: 2.

16. The mutated S glycoprotein of claim 14, wherein the mutated S glycoprotein comprises a sequence at least 95% identical to SEQ ID NO: 2.

17. The mutated S glycoprotein of claim 16, wherein the mutated S glycoprotein comprises SEQ ID NO: 2.

18. A pharmaceutical composition comprising a mutated S glycoprotein of claim 1.

19. The pharmaceutical composition of claim 18, further comprising an adjuvant.

20. A vaccine composition comprising a mutated S glycoprotein of claim 1.

21. The vaccine composition of claim 20, further comprising an adjuvant.

22. A vector comprising a nucleic acid sequence encoding a mutated SARS-CoV-2 S glycoprotein (mutated S glycoprotein) comprising a SARS-CoV-2 S glycoprotein amino acid sequence having one or more mutations compared to a wildtype S glycoprotein, wherein the mutated S glycoprotein minimizes (i) antibody-dependent enhancement (ADE) and/or (ii) vaccine-associated enhanced respiratory disease (VAERD) when expressed in a subject.

23. The vector of claim 22, wherein the mutated S glycoprotein comprises a sequence having at least 70% sequence identity to SEQ ID NO: 1 and one or more of:
(i) a deletion of one or more amino acids from a region spanning amino acid position 14 to amino acid position 32 of a wildtype precursor S glycoprotein of SEQ ID NO: 1;
(ii) a deletion of one or more amino acids from within an N-terminal domain of a wildtype mature S glycoprotein of SEQ ID NO: 2, wherein the deletion removes epitopes in the wildtype mature S glycoprotein that contribute to ADE or VAERD;
(iii) an amino acid substitution at amino acid position 271 in a wildtype precursor S glycoprotein of SEQ ID NO: 1;
(iv) one or more amino acid substitutions in a region spanning amino acid position 611 to amino acid position 616 of wildtype precursor S glycoprotein of SEQ ID NO: 1;
(v) one or more amino acid substitutions in a region spanning amino acid position 682 to amino acid position 685 of wildtype precursor S glycoprotein of SEQ ID NO: 1;
(vi) an amino acid substitution at amino acid position 814 and/or amino acid position 815 of wildtype precursor S glycoprotein of SEQ ID NO: 1;
(vii) an amino acid substitution at amino acid position 986 and/or amino acid position 987 of wildtype precursor S glycoprotein of SEQ ID NO: 1; and
(viii) a deletion of one or more amino acid residues from a region spanning amino acid position 1100 to amino acid position 1273 of wildtype precursor S protein of SEQ ID NO: 1, wherein the deletion promotes transport of the mutated S glycoprotein to a plasma membrane of a mammalian cell.

24. The vector of claim 23, wherein the mutated S glycoprotein comprises a signal sequence.

25. The vector of claim 23, wherein (i) comprises deletion of each of the amino acids in the region spanning amino acid position 14 to amino acid position 32 of a wildtype mature S glycoprotein of SEQ ID NO: 1.

26. The vector of claim 23, wherein (ii) comprises deletion of one or more amino acids from a region spanning amino acid position 24 to amino acid position 270 of a wildtype precursor S glycoprotein of SEQ ID NO: 1.

27. The vector of claim 23, wherein (ii) comprises deletion of each of the amino acids from the region spanning amino acid position 24 to amino acid position 270 of a wildtype precursor S glycoprotein of SEQ ID NO: 1.

28. The vector of claim 23, wherein the amino acid substitution at amino acid position 271 of wildtype precursor S glycoprotein of SEQ ID NO: 1 is selected from the group consisting of: Q271G, Q271A, Q271I, Q271L, Q271M, Q271F, Q271P, and Q271V.

29. The vector of claim 23, wherein the mutated S glycoprotein comprises a contiguous amino acid sequence of LFGSVA (SEQ ID NO: 3) at a region corresponding to amino acid position 611 to amino acid position 616 of wildtype precursor S glycoprotein of SEQ ID NO: 1.

30. The vector of claim 23, wherein the mutated S glycoprotein comprises a contiguous sequence of SGAG (SEQ ID NO: 6) at a region corresponding to amino acid position 682 to amino acid position 685 of wildtype precursor S glycoprotein of SEQ ID NO: 1.

31. The vector of claim 23, wherein the mutated S glycoprotein comprises a contiguous sequence of AN at a region corresponding to amino acid position 814 to amino acid position 815 of wildtype precursor S glycoprotein of SEQ ID NO: 1.

32. The vector of claim 23, wherein the mutated S glycoprotein comprises a contiguous sequence of PP at a region corresponding to amino acid position 986 to amino acid position 987 of wildtype precursor S glycoprotein of SEQ ID NO: 1.

33. The vector of claim 23, wherein the mutated S glycoprotein comprises a deletion of each of the amino acids in a region extending from amino acid position 1255 to amino acid position 1273 of wildtype precursor S glycoprotein of SEQ ID NO: 1.

34. The vector of claim 23, wherein the mutated S glycoprotein comprises a sequence having at least 80% sequence identity to SEQ ID NO: 2.

35. The vector of claim 22 wherein the vector further comprises a promoter operatively linked to the nucleic acid sequence encoding the mutated S glycoprotein.

36. The vector of claim 35, wherein the promoter is a muscle-specific promoter.

37. The vector of claim 36, wherein the muscle-specific promoter is selected from the group consisting of: skeletal β-actin, myosin light chain 2A, dystrophin, muscle creatine kinase, SPc-512, and synthetic muscle promoters.

38. The vector of claim 35, wherein the promoter is selected from the group consisting of: CMV, RSV, SV40, β-actin, PGK, and EF1 promoters.

39. The vector of claim 22, wherein the vector is a viral vector.

40. The vector of claim 39, wherein the viral vector is a lentivirus vector or herpes virus vector.

41. The vector of claim 39, wherein the vector is an AAV vector.

42. The vector of claim 41, wherein the AAV vector comprises an AAV serotype 6 (AAV6) capsid protein.

43. The vector of claim 41, wherein the AAV vector comprises an AAV serotype 9 (AAV9) capsid protein.

44. The vector of claim 41, wherein the AAV vector comprises an Anc80, Anc80Lib, Anc 81, Anc82, Anc83, Anc84, Anc110, Anc113, Anc126, Anc127, or another phylogenetically related AAV capsid protein.

45. The vector of claim 22, wherein the vector is a plasmid.

46. A pharmaceutical composition comprising a vector of claim 22.

47. The pharmaceutical composition of claim 46, wherein the pharmaceutical composition further comprises an adjuvant.

48. The pharmaceutical composition of claim 47, wherein the adjuvant is a CpG adjuvant.

49. A vaccine composition comprising a vector of claim 22.

50. The vaccine composition of claim 49, further comprising an adjuvant.

51. The vaccine composition of claim 50, wherein the adjuvant is a CpG adjuvant.

52. A method of inducing at least partial immunity to a coronavirus in a subject, the method comprising administering to the subject a therapeutically effective amount of a mutated S glycoprotein of claim 1.

53. The method of claim 52, wherein the administering minimizes antibody-dependent enhancement (ADE).

54. The method of claim 52, wherein the administering minimizes vaccine-associated enhanced respiratory disease (VAERD).

55. The method of claim 52, wherein the administering results in at least partial immunity to the coronavirus due to humoral immunity to the coronavirus.

56. The method of claim 52, wherein the administering results in T-cell mediated immunity to the coronavirus.

57. The method of claim 52, wherein the administering results in an increase in titer of antibodies that specifically bind to the mutated S glycoprotein in the subject.

58. The method of claim 52, wherein the administering results in a decrease in the rate of infection of the coronavirus in the subject.

59. The method of claim 52, wherein the method further comprises administering an adjuvant to the subject.

60. The method of claim 59, wherein the adjuvant is a CpG adjuvant.

61. The method of claim 52, wherein the subject has been identified as not having previously had a coronavirus infection.

62. The method of claim 52, wherein, prior to the administering step, the subject has been identified as not having a significant titer of antibodies that bind specifically to the S glycoprotein of the fragment thereof.

63. The method of claim 52, wherein the coronavirus is SARS-CoV-2.

64. The method of claim 52, wherein the subject has been previously identified as having one or more medical conditions selected from the group consisting of: chronic lung disease, moderate asthma, severe asthma, heart conditions, diabetes, obesity, liver disease, chronic kidney disease, and a weakened or suppressed immune system.

65. The method of claim 64, wherein the subject having a weakened or suppressed immune system is a subject receiving a cancer treatment, a smoker, a subject who is a transplant recipient, a subject having HIV or AIDS, or a subject receiving a corticosteroid or any other immunosuppressant drug.

66. The method of claim 64, wherein the subject having a weakened or suppressed immune system is an elderly subject.
